(19) United States
(12) Patent Application Publication
(10) Pub. No.: US 2025/0339472 A1
(43) Pub. Date: Nov. 6, 2025

(54) Mind and Body Healing Synergistic Phytomedical Formulation

(71) Applicant: PetJuvenate Inc., Murrieta, CA (US)

(72) Inventor: Paul G. Moran, Murrieta, CA (US)

(21) Appl. No.: 18/800,890

(22) Filed: Aug. 12, 2024

Related U.S. Application Data
(60) Provisional application No. 63/642,700, filed on May 4, 2024.

(51) Int. Cl. A61K 35/57; A61K 31/122; A61K 31/685; A61K 31/728; A61K 33/32; A61K 36/324; A61K 36/55; A61K 36/899; A61K 36/9066; A61K 38/17; A61K 45/06

(52) U.S. Cl. CPC A61K 35/57; A61K 31/122; A61K 31/685; A61K 31/728; A61K 33/32; A61K 36/324; A61K 36/55; A61K 36/899; A61K 36/9066; A61K 38/1748; A61K 45/06

(57) ABSTRACT

Disclosed in the present invention is a synergistic phytomedical mind and body wellbeing formulation involving a botanical composition with a dual approach to inhibit Cyclooxygenase (COX) and Lipoxygenase (LOX) enzymes that enhances nervine function for therapeutic intervention encompassing phytoceutical compositions and methods for treating inflammatory conditions, neurological and gut microbiome disorders. The formulation integrates a selected blend of natural ingredients with main active ingredients, including Stabilized Flax Meal, Cynatine® FLX, Sunflower Lecithin, Boswellia Extract, Turmeric/Curcumin C-3 Reduct®, Egg Shell Membrane, Green Oat Extract-Neuravena®, Hyaluronic Acid, Manganese Chelate and AstaReal® Astaxanthin are carefully chosen for their documented efficacy in addressing various dimensions of chronic pain and inflammation, as well as promoting joint/tissue health, gut health and cognitive function. Through synergistic action, these ingredients work in tandem to provide holistic relief from chronic inflammation, thereby improving quality of life without reliance on harsh pharmaceutical interventions.

### TECHNICAL FIELD

The present invention relates to pharmacology and state of wellness, and more specifically, to a mind and body wellbeing phytomedical formulation, and comprises of a synergistic botanical formulation with a dual approach to inhibit Cyclooxygenase (COX) and Lipoxygenase (LOX) enzymes while enhancing nervine function for therapeutic intervention, encompassing pharmaceutical compositions and methods for treating inflammatory conditions and neurological disorders, comprising preparing the body for accelerated healing by facilitating a much calmer, stress reduced host, and consequently the ingredients can be effective with less interference from the body's own defenses to pain and inflammation using naturally derived ingredients to calm the mind and body.

### BACKGROUND ART

Converging evidence from various researchers has shown that psychological stress and other behavioral factors can affect healing. Stress-induced glucocorticoid production has been associated with delayed healing, as has increased catecholamine production. In pharmacy, pharmacological effects of one constituent are usually enhanced by one, or more constituents in any given formula. The nature of such synergistic effect is probably resulting from the bioactive ingredients acting on either the same receptor target, or a similar physiological system.

In one prior art by Lindsay K. Caesar and Nadja B. Cech (2019) discloses a synergy and antagonism in natural product extracts. It provides a critical review with commentary about the current state of the scientific literature as it relates to studying combination effects (including both synergy and antagonism) in natural product extracts. Guidance by the natural products community is needed to provide strategies for effective evaluation of safety and toxicity of botanical mixtures and to drive discovery in botanical natural product research.

In another prior art by Ilya Raskin and Christophe Ripoll (2004) describes how an apple a day can keep the doctor away. Multi-component botanical therapeutics that comprise functional foods, dietary supplements and botanical drugs hold several advantages over conventional drugs that may earn them a more prominent place in the medicine of the future.

They can deliver mixtures of multi-functional molecules with potentiating and synergistic effects and pleiotropic targeting at a reasonable cost and with fewer regulatory constraints. They are well suited for long-term disease prevention in an era of genetic testing and increased life expectancy.

Thomas Effort and Egon Koch (2011), Pub Med describes a complex interaction between phytochemicals. The multi-target therapeutic concept of phytotherapy. Presents mechanistic reasons for interactions are bioavailability, interference with cellular transport processes, activation of pro-drugs or deactivation of active compounds to inactive metabolites, action of synergistic partners at different points of the same signaling cascade (multi-target effects) or inhibition of binding to target proteins. “Omics” technologies and systems biology may facilitate unravelling synergistic effects of herbal mixtures.

Sandy van Vuuren 1, Alvaro Viljoen (2012), Planta Med discloses a plant-based antimicrobial studies—methods and approaches to study the interaction between natural products. This paper intends to provide an overview, from an antimicrobial perspective, on the research undertaken and interactive principles involved in pharmacognosy studies. Methods used to determine antimicrobial interactions include basic combination studies, the sum of the fractional inhibitory concentration index (EFIC), isobole interpretations, and death kinetic (time-kill) assays.

Chronic pain is characterized by persistent discomfort lasting for an extended period, often beyond the expected time for tissue healing. It can derive from various underlying conditions, including arthritis, fibromyalgia, neuropathy, and musculoskeletal disorders. In addition to physical discomfort, chronic pain is often associated with cognitive impairments such as reduced attention, memory problems, and impaired decision-making. Furthermore, the psychological impact of chronic pain, including anxiety, depression, and decreased quality of life, further exacerbates the burden of the condition on affected individuals.

Traditional approaches to chronic pain management typically involve the use of analgesic medications, nonsteroidal anti-inflammatory drugs (NSAIDs), and opioids. While these medications may provide temporary relief, they often come with a host of side effects and risks, including dependency, tolerance, and addiction. Moreover, they do not address the underlying causes of chronic pain or its associated cognitive and emotional symptoms.

Mastering herbal synergy can lead to a plethora of benefits, from boosting your immune system to treating various health conditions. Some well-established synergistic combinations include: Turmeric and Black Pepper (increases the bioavailability of Curcumin), Echinacea and Goldenseal (increases immune support), or Curcumin and Boswellia (reducing oxidative stress in athletes) Lecithin and Omega 3 Fatty Acids (Help the Fatty Acids become more Bioavailable, and works in a similar fashion with other novel ingredients like CBD and more) Manganese is essential in chemical processes that allow your body to use Nutrients and Vitamins such as Choline, Thiamine, Vitamin C and Vitamin E and various other known examples.

In recent years, there has been a growing interest in exploring alternative and complementary therapies for chronic pain management, with a particular focus on natural ingredients with anti-inflammatory, antioxidative, and nervine properties. These botanical ingredients offer the potential to provide holistic relief from chronic pain while minimizing the risks associated with pharmaceutical interventions. By targeting multiple pathways involved in pain perception, inflammation, and cognitive function, synergistic botanical formulations hold promise as a safe and effective approach to chronic pain management.

Katherine H M Cox, Andrew Pipingas, and Andrew B Scholey (2014), Journal of Psychopharmacology describes an investigation of the effects of solid lipid Curcumin on cognition and mood in a healthy older population. Curcumin possesses many properties which may prevent or ameliorate pathological processes underlying age-related cognitive decline, dementia or mood disorders. These benefits in preclinical studies have not been established in humans. This randomized, double-blind, placebo-controlled trial examined the acute (1 and 3 h after a single dose), chronic (4 weeks) and acute-on-chronic (1 and 3 h after single dose following chronic treatment) effects of solid lipid Curcumin formulation (400 mg as Longvida®) on cognitive function, mood and blood biomarkers in 60 healthy adults aged 60-85. In one prior art by S. K. Kulkarni and A. Dhir (2010) in an Overview of Curcumin in Neurological Disorders, Curcumin does not have the potential antidepressant effect of desipramine (tricyclic antidepressant and norepinephrine reuptake inhibitor) and imipramine (tricyclic antidepressant)[1]. This gives an indication of the involvement of serotoninergic and dopaminergic system in the antidepressant action of Curcumin. When the brain neurotransmitter levels were checked following Curcumin administration, it increased the levels of serotonin and dopamine but not norepinephrine in the mouse brain [1].

The US patent application that was published as U.S. Pat. No. 6,544,563B2 in 2003 describes a method and composition for improving sexual fitness. The invention provides methods and compositions for maintaining a state of wellness in a human by providing a dietary supplement comprising L-arginine, alone or in combination with Ginseng and Ginkgo biloba and/or additional nutritional supplements. The invention provides a unique blend of components that, in combination, synergistically bestow sexual wellness upon a human when taken regularly as a dietary supplement.

Behdad Jahromi et al (2021) discloses a review of herbal medicine for pain management: efficacy and drug interactions. The review compiles common and available herbal medicines which can be used as an alternative to or in combination with conventional pain management approaches. Efficacy and safety are assessed through clinical studies on pain relief. Ensuing herb-drug interactions such as cytochrome modulation, additive and synergistic effects, and contraindications are discussed. While self-management has been recognized as part of the overall treatment strategy for patients suffering from chronic pain, it is important for practitioners to be able to also optimize and integrate herbal medicine and, if warranted, other complementary and alternative medicines into their care.

The US patent application number U.S. Pat. No. 8,062,680B2 of 2010 discloses a synergistic phytoceutical compositions for the prevention and treatment of circulatory disorders, feminine endocrine disorders, and dermal disorders. A specific combination of extracts of plants is taught, as well as principles for varying the formulations based on categorizing plants into one of three groups, Energy, Bio-Intelligence, and Organization and selecting several plants from each group. Such combinations have synergistic effects, with minimal side effects.

Currently, there is no pet mobility formula that addresses the joint & tissue support with mental wellbeing/mood support (not just cognitive support) simultaneously. It is clinically proven that being injured and in pain creates a subdued and sometimes depressed state of mind. Our intention is to improve the mental state, create a plant based chemically induced “window” that allows a synergistic approach to the other anti-inflammatory/anti-oxidative ingredients to go to work with reduced clucocorticoid/catecholamine interference and an increase in dopamine and serotonin production, all botanically induced.

Similar formulations are found in the pet industry. Specifically, the 8 Out of 10 Dog. owners that will be affected by Osteoarthritis (OA) and mobility issues in their older dogs, during their lifetime.

But all these are different what is being proposed in the present invention.

Quality of life is increasingly valued in today's society. Proper nutrition and exercise, and healthy body functionality contributes to maintaining an overall state of wellbeing, which can serve to manage stress, maintain a properly performing immune system, protect against disease, maintain a positive mental outlook, and generally to enable one to feel good and enjoy life.

At the moment there is no pet mobility formula that addresses the joint & tissue support, anti-inflammation/anti-oxidant support, mental wellbeing/mood support (not just cognitive support) and gut microbiome support, simultaneously. It is clinically proven that being injured and in pain creates a subdued and sometimes depressed state of mind. Research has proven that reducing stress promotes faster healing. The present invention intents to improve the mental state, create a plant based chemically induced “window” that allows a synergistic approach to the other anti-inflammatory/anti-oxidative ingredients to go to work with reduced glucocorticoid/catecholamine interference and an increase in GABA (Gamma-aminobutyric acid), dopamine and serotonin production, all botanically induced to lay the foundation for a more receptive and accelerated healing process.

Therefore, there is dire need to provide a mind and body wellbeing synergistic phytomedical formulation that can contribute the “Mind and Body” wellbeing of both dogs and indeed, humans too.

### OBJECTS OF THE INVENTION

The primary object of the invention is to provide a novel synergistic botanical formulation capable of inhibiting Cyclooxygenase (COX) and Lipoxygenase (LOX) enzymes concurrently, thereby offering a dual approach to attenuating inflammation.

The second object of the present invention is to enhance the efficacy of therapeutic interventions by incorporating botanical extracts with proven COX and LOX inhibition properties, thereby potentially reducing the reliance on single-target pharmaceuticals with associated side effects.

The other object of this invention is to mitigate inflammation-related pain and discomfort through a synergistic combination of botanical ingredients targeting multiple pathways involved in the inflammatory cascade addressing both the brain and microbiome (second brain) the CNS and ENS respectively.

The following summary is an explanation of some of the general inventive steps for the system, method and procedures in the description.

### SUMMARY OF THE INVENTION

The present invention describes a novel mind and body wellbeing synergistic botanical formulation designed to address inflammation through a dual approach, inhibiting Cyclooxygenase (COX) and Lipoxygenase (LOX) pathways while simultaneously enhancing nervine function and destressing the host in order to accentuate the healing process.

The invention combines known ingredients in a not previously introduced format to have a new and specific dual approach to pain and inflammation in dogs. It has addressed the anti-inflammation support and the equally important stressed mental state caused by chronic pain, which research has proven causes a slower healing process.

The invention is designed to fight the mental stress caused by injury (pain and inflammation), improving mental state and mood (wellbeing) as a precursor and equal partner in faster, more targeted healing while incorporating a synergistic attack on inflamed joints and tissues and all with a non-pharmaceutical, natural botanically derived delivery vehicle.

The present invention provides a powerful anti-inflammatory, gut health and mental wellbeing supplement. It is addressing anti-inflammatory COX/LOX pathways, discomfort, microbiome health, muscle recovery, cartilage remodeling, but in addition addressing “mental/cognitive” state (anti-anxiety) with the use of Green Oat Extract—Neuravena®, Sunflower Lecithin, Flaxseed Meal, Boswellia and Astaxanthin—AstaReal® creating an omni-directional and synergistic approach to lessening the overall anxiety and distress caused by chronic pain, and allowing the nervous system (brain and gut) to literally relax, be less stressed and let the powerful and clinically tested anti-inflammatory ingredients go to work. The opens the door to an improved and accelerated natural approach to utilizing mostly herbs/plant-based ingredients to improve quality of life, with a non-pharmaceutical approach. Novel and ancient yet efficacious ingredients have been utilized.

In a preferred embodiment, the invention describes development of botanical formulations capable of simultaneously inhibiting Cyclooxygenase (COX) and Lipoxygenase (LOX) enzymes, while also enhancing nervous system (CNS and ENS) function. It involves the integration of herbal medicine, pharmacology, and neurology to create novel therapeutic interventions for conditions such as inflammation-related pain and neurodegenerative diseases.

By combining active ingredients such as Stabilized Flax Meal, Boswellia extract, Hyaluronic Acid, Curcumin C-3 Reduct®, Cynatine® FLX and Eggshell Membrane (ESM). The formulation effectively targets physical symptoms like pain/discomfort, muscle recovery, and cartilage remodeling.

Additionally, and the key to the synergistic effect of the formulation, is the inclusion of Green Oat Extract-Neuravena®, Sunflower Lecithin, Curcumin C-3 Reduct®, Flaxseed Meal, Boswellia, and Astaxanthin-AstaReal® that addresses the mental and cognitive states, providing anti-anxiety effects that reduce the instances of immobilizing distress associated with chronic pain. This holistic approach offers a non-pharmaceutical solution to improve quality of life, harnessing the power of natural ingredients to promote overall health and well-being with a multi-channel, whole-body approach to preparing the host for accelerated healing, by addressing and preparing both the Mind . . . and the Body to heal simultaneously.

Basically, the invention prepares the body for accelerated healing by enabling a much calmer, stress less host, so the ingredients can be effective with less impedance from the body's own defenses to pain and inflammation. In fact, it involves complete “mind-body healing” (common in ancient medical modalities) using naturally derived ingredients to calm mind and body as applicable. In addition, the formulation promotes using a high content of stabilized Flaxseed Meal (a natural prebiotic) to specifically address the needs of the “second brain” (the gut microbiome) as it relates to de-stressing the whole body, and other beneficial factors. The gut is packed with over 100 million nerve endings that communicate with the brain and it is sometimes referred to as the “gut-brain axis.” Studies have suggested a link between gut bacteria and disorders of the CNS (central nervous system) like anxiety, depression, and even ASD (autism spectrum disorder).

Scientists are increasingly finding the important roles gut microbiota plays in your overall health, from diabetes to heart disease. The gut microbiota helps to regulate the function of the gut-brain axis. The microbiota and brain communicate with each other on several levels, the ENS (enteric nervous system), the Vagus Nerve (direct brain communication link) and other pathways. In fact, 90% of the neurons in the Vagus Nerve are carrying information from the gut to the brain, not vice versa. Meaning the gut generates signals that have a hugely important influence in the brain. We embrace this in the belief that our formula can increase the production Serotonin and Dopamine (documented later) which creates a feeling of wellbeing in and of themselves but, also of GABA (a neurotransmitter that triggers calming) and research has shown some of the properties of these ingredients can even signal neurons to release GABA which acts on the Vagus Nerve and has been shown to reduce inflammation and stress. Neuroplasticity, a process involved with mood creation is also directly influenced by microbes in the microbiome.

A major support pillar of this formulation is that we address and recognize that gut health is an important part overall health and wellness and that gut health is affecting mental and physical health in so many ways. With more exciting research ongoing, and new discoveries happening on a regular basis, we are delighted to be a proponent of “second brain health” being an extremely integral and irrefutable function in overall wellness. We have deliberately addressed this as an important element in our formulation to help reduce both physical and mental stressors thus helping to eliminate what were previously roadblocks to a faster and synergistically induced, multi-channel directed state of Mind and Body wellbeing.

There is currently no pet product in the marketplace that offers the real benefits, objectives and the synergistic, specific multi-channel approach to improved mind and body wellbeing, as evidenced in the PetJuvenate® formulation.

The above summary is not an extensive overview of the invention and does not intend to limit the scope beyond what is described and claimed as a summary.

### BRIEF DESCRIPTION OF THE DRAWINGS

FIG. 1 is a representation of a mind and body wellbeing synergistic phytomedical formulation as described in the present invention.

FIG. 2 is the schematic steps of formulating and administering the mind and body wellbeing synergistic phytomedical formulation as described in the present invention.

### DESCRIPTION OF THE INVENTION

In order to explain the technical contents, the achieved objects, and the effects of the present application in detail, the following description is made in conjunction with the embodiments and the accompanying drawings.

In the following detailed description of the invention, numerous details, examples, and embodiments of the invention are described. However, it will be clear and apparent to those knowledgeable in the field that the invention is not limited to the embodiments set forth and that the invention can be adapted for a wider range of applications.

In this description, the terms “comprising,” “including,” “containing,” “characterized by,” and grammatical equivalents thereof are inclusive or open-ended terms that do not exclude additional, un-recited elements or method acts, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

The singular forms “a,” “an,” and “the” are intended to include the plural forms as well, unless the context clearly indicates otherwise. The term “and/or” includes any and all combinations of one or more of the associated listed items. As used herein, relational terms, such as “first,” “second,” “top,” “bottom,” “upper,” “lower,” “over,” “under,” etc., are used for clarity and convenience in understanding the disclosure and do not connote or depend on any specific preference, orientation, or order, except where the context clearly indicates otherwise.

The research of Bruce McEwen, head of the neuroendocrinology lab at Rockefeller University (New York, NY, USA) has also shown that stress hormones have dual effects on the brain-protective in the short term, but damaging in the long term by impairing nerve cells in certain areas of the brain. He developed the concept of allostatic load—damaging changes that can accumulate in response to stress because the overexposure to neural, endocrine and immune stress mediators has adverse effects on various organ systems.

Chronic activation of stress responses by the hypothalamic-pituitary-adrenal axis and the sympathetic-adrenal-medullary axis leads to a permanent overproduction of glucocorticoid hormones and catecholamines (adrenaline and noradrenaline).

The terms that are being utilized are Naturopathic or Phytomedical approach to Mind-Body wellbeing and healing. For example, in relation to Green Oat Extract—Neuravena® studies have shown this ingredient has powerful Phytomedical and Neuroprotective benefits. Oat Straw (Avena sativa) as a nootropic is an extract produced from the common oat plant while still in its milky stage. Oats are a cereal grain grown in temperate climates world-wide as food for humans and livestock, and as an ingredient used in cosmetics. The oat plant is descended from Avena sterilis, a wild oat that originated in the ‘Fertile Crescent’ that spanned from Israel to western Iran to Europe.

It was domesticated about 3,000 years ago in Europe. Oat straw increases cognition Oat Straw (Avena sativa). Tea brewed from oats has traditionally been used as a mild sedative, and a herbal treatment for anxiety, insomnia and for some nervous type disorders. Avenanthramides (bioactive compounds unique in oats) have been shown to enhance nitric oxide (NO) production in human smooth muscle cells and have anti-inflammatory, anti-proliferative, and anti-itching activity.

Green Oat Extract inhibits phosphodiesterase type 4 (PDE4). PDE4 is a component of signaling pathways involved in the mediation of antidepressant activity. PDE4 inhibitors can prolong the effects of cAMP in the brain which can improve long-term memory, wakefulness, is neuroprotective, works as an anti-inflammatory and antidepressant. Green Oat Extract inhibits monoamine oxidase B (MAO-B) which increases dopamine levels in the brain. Helping brain disorders like ADHD and Parkinson's Disease. Green Oat Extract suppresses inflammatory cytokines by inhibiting nuclear factor κB (NF-κB) activation[viii] Cytokines are implicated in a number of brain disorders including major depression, schizophrenia and Alzheimer's Disease. It is one of the building blocks to our “mind-body phytomedical formulation approach.

In one prior art by Jianling Liu, Fengxia Shen et al (2018) in a Scientific Reports Volume 8, Article number: 380 (2018) discloses “Systems pharmacology analysis of synergy of TCM: an example using Saffron formula”. The report cites that Traditional Chinese medicine (TCM) follows the principle of formulae, in which the pharmacological activity of a single herb can be enhanced or potentiated by addition of other herbs. Nevertheless, the involved synergy mechanisms in formulae remain unknown. Here, a systems-based method is proposed and applied to three representative Chinese medicines in compound Saffron formula (CSF): two animal spices (Moschus, Beaver Castoreum), and one herb Crocus sativus which exert synergistic effects for cardiovascular diseases (CVDs).

From the formula, 41 ingredients and 66 corresponding targets are acquired based on the ADME evaluation and target fishing model. The network relationships between the compounds and targets are assembled with CVDs pathways to elucidate the synergistic therapeutic effects between the spices and the herbs. The results show that different compounds of the three medicines show similar curative activity in CVDs.

Additionally, the active compounds from them shared CVDs-relevant targets (multiple compounds-one target), or functional diversity targets but with clinical relevance (multiple compounds-multiple targets-one disease). Moreover, the targets of them are largely enriched in the same CVDs pathways (multiple targets-one pathway). These results elucidate why animal spices and herbs can have pharmacologically synergistic effects on CVDs, which provides a new way for drug discovery.

However, the formulation being disclosed in the above prior art differs in both form and use from that being described in the present invention.

The present invention encompasses a synergistic botanical formulation carefully crafted to address multiple aspects of inflammation and nervous system function. The formulation incorporates a combination of active ingredients derived from natural sources, each chosen for its specific and proven therapeutic properties.

The invention formulated to help enhance the healing process we propose that by utilizing a dual action, botanical approach to treatment of inflammation in the body, by reducing nervine stressors and stimulate a natural and normal release of glucocorticoids (not over-production), to create a “window of healing” that allows the formulations known/proven anti-inflammatory ingredients to work synergistically in a less stressed, calmer host, absent of the nervous system hyper activation, hyper anxiety and lowered plasma oxytocin and vasopressin levels, proven to slow down the healing process considerably. Utilizing the various botanical ingredients, it is also possible to increase GABA, Serotonin and Dopamine levels, which in turn creates a less stressed host with a system that is now more receptive to whole body healing.

### Analysis and Hip and Joint Mobility Soft Chews.

The present formula is not typical of conventional hip and joint supplements that we routinely encounter in the marketplace. It is a detailed analysis of the formula and the findings that are promising. Novel and ancient yet efficacious ingredients have been used in the product. The mobility product is developed with a proprietary formula that combines premium human grade ingredients that offer an alternative to Glucosamine in light of recent studies that have reported mixed results on actual efficacy, and also to facilitate dogs that may be “shellfish sensitive” or have full blown shellfish allergies. The invention produces a clean, natural product and formulation.

Each 4 g soft chew-consists of the following active ingredients: Stabilized Flax Meal 400 mg, Cynatine® Flx 150 mg, Turmeric 50 mg—Curcumin C-3 Reduct® (Tetrahydrocurcuminoids), Sunflower Lecithin 80 mg, Boswellia Extract 50 mg, Egg Shell Membrane 50 mg, Green Oat Extract—Neuravena® 50 mg, Hyaluronic Acid 5 mg, Manganese Chelate 3 mg and Astaxanthin—AstaReal® 2 mg.

Flaxseed is a nutrient rich ingredient. It is considered as a “Superfood” for its many bioactive compounds present in it that promote optimum health. To that point, Flaxseed is one of the richest sources of plant derived Omega 3 Fatty acids. Flaxseed is a natural anti-inflammatory and bolsters the immune system. It is also a natural prebiotic, which is essential for gut microbiome health (Second Brain Health) and improved digestion, which in itself is a “whole system” de-stressor.

Stabilized Flaxseed meal has a longer shelf life and therefore retains more nutrients long term, than its non-stabilized form. This is why this formulation calls for the stabilized form of flaxseed. Flaxseed meal is also the most bioactive form and allows the maximum absorption rate of nutrients possible, compared to whole Flaxseeds or Flaxseed oil. The anti-inflammatory, anti-oxidant, anti-proliferative properties and the high fiber content of Flaxseeds provide many health benefits.

Flaxseed meal contains Thiamine, Vitamin B-6, Copper, Manganese, Phosphorus, Zinc Iron, Selenium, Magnesium and Folate. Flaxseed meal has high thiamine (Vitamin B1) content and contributes an important role in energy metabolism as well as cell function. It's also a solid source of copper, which is vital for brain development, the metabolism of iron in the body, and overall immune health.

### Potential Biochemical and Metabolic Action

The majority of the nervous system is composed of fat. Cell to cell signaling in the brain occurs via the sacrolemmal membrane and is transmitted through membranes of fatty acids, which exemplifies the importance of fatty acids in the overall function of the brain. Alpha Linoleic Acid (ALA) is an essential Omega 3 fatty acid. The ingestion of ALA has shown to increase the speed of transmitting signals. It has been found out that sufficient levels of Omega 3 fatty acids are not produced by the body and needs to be incorporated through the diet to keep the signals moving smooth. This formula utilizes the universally recognized “Super food”, Flaxseed Meal, which is one of the richest sources of Omega 3 fatty acids available from plant sources.

According to Al-Madhagy et al., 2023 flaxseed yields 35-45% of oil out of which more than 70% is ALA. It has been shown that Omega 3 lessens mental fatigue in chronic low-grade inflammation, as demonstrated in obesity (Gholami Z and Akhlaghi, 2021). When flaxseeds are ingested, the linoleic acid (LA) in the Flaxseeds is converted to Arachidonic acid and ALA is converted to eicosapentaenoic acid (EPA) and docosahexaenoic acid (DHA). Arachidonic acid acts as the progenitor of Prostaglandin E (PGE) and Thromboxane A2 via the Cyclooxygenase (COX) pathway. Arachidonic acid is also the precursor of Leukotriene A4 (LTA4) to Leukotriene B4 (LTB4) conversion via the 5-Lipooxygenase (5-LOX) pathway (Al-Madhagy et al., 2023). The COX and the LOX pathways are the two main pathways that produce pain and inflammation in the body. The inhibition of the COX and the LOX pathways contribute to reduced pain and reduced inflammation, thus promote less-stressful healing. On the other hand, EPA acts as the substrate for 5-LOX through Leukotriene B5, resulting in a mild inflammatory activity (Al-Madhagy et al., 2023).

ALA is responsible for reducing the production of Inflammatory Cytokines, Lipids and Lipokinins and also inhibits pro-inflammatory cytokine production, resulting in the stimulation of osteoblasts to release osteoclast factors and up regulate the proliferation and activation of osteoclasts. These processes promote bone resorption in the body and reducing pain and inflammation. Flaxseed oil provides neuroprotection by the activation of brain derived neurotrophic factor and glial cell derived neurotrophic factor (Al-Madhagy et al., 2023). Both EPA and DHA are essential components of cell membranes that are critical for brain function and mood fixing. Omega 3 is proven to reduce cortisol levels in the body. Studies show promising effects of Flaxseed Oil in the treatment of depression and anxiety, demonstrating improved cognition, reduced mental stressors and pain generation.

Flaxseed Oil is also known for its anti-inflammatory activity. Flaxseed Oil releases anti-inflammatory lipid mediators and anti-inflammatory peptide mediators. The lipid mediators include Prostaglandin E3 (PGE3)—a product of EPA by the COX pathway, PGE2 and LTB4 and the peptide mediators include cytokines, interleukins, superoxide, histamines and enzymes. Resolvin and Pectin are produced by DHA by the LOX pathways (Al-Madhagy et al., 2023). Thus it is clear that both the LOX and the COX pathways are activated and a synergistic effect is produced. The Moss Center for Intergrative Medicine recommends Flaxseed Powder as an essential component in the daily diet (Moss Center For Intergrative Medicine, 2018). The Lignins in Flaxseed are known to reduce the formation of atherosclerosis plaques by reducing the oxidative stress and by regulating the blood cholesterol and LDL levels.

Cynatine® FLX is a bioactive and soluble form of Keratin protein. Keratin has a high sulphur content and is almost entirely in the form of the amino-acid, cystine. No other protein is so high in Cystine. Human joints are vulnerable to the effects of ageing due to the presence of oxidative stressors and constant wear and tear. To compensate the effects of various forces that degrade joints, scientists in New Zealand formulated a novel molecule comprising of a soluble form of Keratin protein that is bioactive, which comes to us as Cynatine® FLX. It is a “force multiplier” for the natural defense of the body.

Randomized research shows the efficacy of Cynatine® FLX on symptoms of osteoarthritis by reducing pain scores by twice as compared to placebo, and shows the same efficacy in reducing stiffness. Solubilized Keratin rebuilds vital joint tissues with essential bio active high Sulphur precursors. The anti-inflammatory properties and the anti-oxidant properties combine to promote overall joint health. Ingesting Cynatine® FLX produces increased production of superoxide dismutase and glutathione—a major anti-oxidant in the body. The increased levels of glutathione dependent peroxidase help as the primary defense against oxidative damage. The anti-oxidant property of solubilized Keratin increases mobility of joints by reducing the damage in joints caused by free radicals and by reducing pain in the joints. Similarly, the use of solubilized Keratin shows improved skin health by increasing skin moisture, skin elasticity and appearance

### Potential Biochemical and Metabolic Action

The main biologically active component of Cynatine® FLX is Keratin. In 2013 C. Beer et al studied the clinical efficacy of Cynatine® FLX and confirmed that it may be effective as a novel therapeutic agent in the treatment of osteoarthritis related joint pain. Wool Keratin showed a quick response in the treatment of initial stages of osteoarthritis (Gallucio, Barskova and Cerinic, 2015). Keratin based material stimulate the production of PGE2 by inhibiting Interleukin-1, thus reducing joint inflammation. This highly desirable property in Cynatine® FLX makes it an effective therapy in joint pain. Diseased joints have compromised sulfur metabolism and less strength. Wool Keratin has a higher percentage of Cysteine in the form of the dimer Cystine, when compared to the percentage of the rest of the amino acids present in wool Keratin, thus the ability to improve the joints sulfur metabolism and overall health.

Cysteine, Homocystein, Methionine and Taurine are the four main amino acids containing sulfur bonds. The water-soluble fraction of Keratin includes several proteins and peptides. The derivatives of these proteins and petides include di-sulfide bonds that get converted to S-sulfonated bonds and sulfonic acid groups by oxidation. The S-sulfo group is a derivative of cysteine and when S-sulfo cysteine groups react with reducing agents such as cysteine and thiols, cysteine is formed once again by readily forming di-sulfide crosslinks (Kelly et al., 2006). The high number of sulfur bonds in the cysteine bridges of Keratin depicts a weight bearing property that is a helpful characteristic in joints to act as a shock absorber. Keratin upregulates osteogenic markers type 1 collagen alpha 1, runt related transcription factor 2, and vitamin D receptor and chondregenic markers such as SRY-box 9. These further suggest the ability of Keratin to aid in osteogenesis and chondrogenesis (Wu et al., 2015) This property is applied into the joints to support joint mobility, joint resilience and joint structure. Keratin extract contain growth factors such as Cytokines, Bone Morphogeneic Protein-4 (BMP) and Transforming Growth Factor-Beta that further aid in bone regeneration.

Cysteine upregulates the production of Taurine from cysteine by increasing cysteine diooxygenase levels. Taurine scavenges free radicals produced by hypochlorous acid and forms Taurine-C1 which is an anti-inflammatory mediator to reduce joint inflammation (Kelly et al., 2006). The disulfide bonds present in cysteine aid in resistance and stability (Giteru et al., 2022). Proteoglycans provide flexibility and resistance to the joints. They are naturally present in the joints and to maintain the function of proteoglycans in the joints the proteoglycans will be naturally highly sulfated. As mentioned above, the joints degrade with less sulfur bonds and it has been found that the degrading sulfur levels can be compensated by the ingestion cysteine rich sources such as Keratin as the cysteine is catabolized by cysteine dioxygenase.

In 2004, Kelly, Roddick-Lanzilotta and Ali received the patent for a product aimed at healing wounds using wool Keratin (Kelly, Roddick-Lanzilotta and Ali, 2004). The sulfur bonds in the intermediate protein filaments in the core of Keratin structures have been utilized to produce this invention (U.S. Pat. No. 7,732,574B2). Keratinocytes contain Keratin that supports Keratinocytes to migrate and re-form epithelial tissues and support the up regulation of collagen. These processes are important in tissue healing (Ranjit et al., 2022). Cynatine® FLX is rich in cysteine and glutathione which also confirms its suitability to act as a protein supplement to increase lean body mass. The increased supply of bioactive peptides and amino acids by Cynatine® FLX accelerate wound healing, confirming that the product promotes a window to host system healing, through effective use of this synergistic formulation.

Keratin is the richest natural source of cysteine. It has been found that deficiency in dietary cysteine and low hepatic glutathione concentrations increase oxidative stress, growth retardation and an increased vulnerability to age related degenerative processes. It was found by Ignowski et al, 2018 that cysteine supplementation significantly increased glutathione levels. Keratin acts as an anti-oxidant by stimulating several enzymes to inhibit oxidative stress and Keratin itself possesses an intrinsic anti-oxidant effect. Cynatine® FLX also contributes widely to the anti-oxidant and anti-inflammatory effects of the body system. Malondialdehyde is a biological marker of oxidative stress. Cynatine® FLX produces its anti-oxidant and anti-inflammatory effect by reducing the concentrations of malondialdehyde and boosted glutathione and other sulfur-rich proteins and peptides.

In 2006, a nutraceutical oral supplement derived of Keratin and Keratin soluble material received a patent; Patent Number WO2006099309A2, for its anti-oxidation and anti-inflammation abilities, and promoting skin health. The formula goes beyond this effect to not just produce anti-oxidative and an anti-inflammatory effect but with an extra effect on calming the nervous system through the various metabolic mechanisms noted. This patent also reveals the safety of the use of Keratin and Keratin soluble material in the form of an oral supplement as a nutraceutical at the dose rate of 10 mg-35 mg per day in veterinary. Several other safe dose rates have been suggested to address various other therapeutic effects Turmeric/Curcumin-C3 Reduct® (White Curcumin extract w/ Tetrahydrocurcuminoids THC's)

Turmeric is an ancient herbal medicine that has been used through many generations as an anti-inflammatory and an antioxidant ingredient. In PubMed, and as of this writing, turmeric has almost 7,000 papers and Curcumin, almost 20,000 papers. In most of the South Asian countries' turmeric is used as a daily spice in food for its proven health benefits. Turmeric regulates proinflammatory enzymes Cyclooxygenase (COX), Lipoxygenases and regulates the inflammatory transcription factors Nuclear Factor kappa B (NF-kB) and Signal Transducer and Activator of Transcription 3 (STAT3) thus, producing an anti-inflammatory effect in the body's entire system. https://pubmed.ncbi.nlm.nih.gov/37082752/Potential Biochemical and Metabolic Action:

The “Wonder Drug of Life” is what turmeric is known as (Gera et al., 2017). The most common active ingredient of turmeric is “Curcumin” and it is recognized as a Generally Regarded as Safe (GRAS) material that is metabolically stable and of low toxicity.

Tetrahydrocurcumin is a derived from Curcumin through process of hydrogenation. It is superior to Curcumin as it has increased chemical stability, increased antioxidant activity, much greater bioavailability. In addition, it has anticancer properties supplied through inherent processes like modulation of oxidative stress, inflammation, proliferation, metastasis, xenobiotic detoxification, immunity and even programmed cell death.

The difference in their pharmacokinetics is that tetrahydrocurcumin does not produce reactive oxygen species due to the absence of conjugated bonds in the central chain whereas Curcumin tends to produce reactive oxygen species and may elicit a prooxidant effect at higher doses. Studies have found that the activity of Curcumin and tetrahydrocurcumin are distinct from each other. Tetrahydrocurcumin is more potent but has fewer anti-inflammatory effects than when compared to the effects of Curcumin but due the less bioavailability effect of Curcumin has necessitated the presence of tetrahydrocurcumin and hence its use in this formulation.

Turmeric/Curcumin produces its anti-oxidative and anti-inflammatory effects in various ways. The anti-oxidative effect is produced by scavenging radicals such as Nitric Oxide, Hydrogen peroxide and superoxide, by enhancing oxidative enzymes such as Superoxide Dismutase, CAT, GPX, OH-1 and thereby inhibit lipid peroxidation, increase glutathione by up regulation of GSH-transferase and their mRNAs, inhibit Reactive Oxygen.

Species producing enzymes such as COX, LOX, Xanthine Oxidase and scavenge peroxyl radicals (Sharifi-Rad et al., 2020). The anti-inflammatory effect is produced by the inhibition of pro inflammatory transcription factors NEkB, AP-1, reduction of pro inflammatory cytokines TNF-alpha, IL-1b, IL-2, IL-6, IL-8, MIP-1a, MCP-1, CRP, PGE 2, down regulation of enzymes 5-Lipoxygenase, COX-2, COX-5, inhibition of nitrogen activated protein kinases and inhibition of pathways involved in Nitric Oxide synthase synthesis (Sharifi-Rad et al., 2020; Moudgil and Venkatesha, 2022) by the upregulation of CD36 and the double gene expression of FOXO3a activity.

In contrast, tetrahydrocurcumin does not upregulate the CD36 and FOXO3a expression and is more effective in its action in TNF-alpha induced ROS production. Studies show that Curcumin is able to inhibit the induction of COX-2 gene expression. Several in vitro studies have demonstrated the potent antioxidant activity of tetrahydrocurcumin in comparison to that of Curcumin to evaluate the activity of scavenging free radicals

Neuro inflammation is a chronic inflammation that occurs by activating the microglia and astrocytes to produce metabolic changes in neurons. These metabolic changes lead to neuronal degeneration and ultimately neuronal death. Curcumin produces neuro-protection in several effective ways. Curcumin inhibits the production of inflammatory cytokines and prostaglandins in activated microglia and astrocytes, inhibit the production of TNF-alpha, certain interleukins, macrophage-inflammatory protein, monocyte chemo-attractant protein in microglial and astrocyte cells and modulate several targets such as transcription factors, enzymes such as COX, LOX, NO and XO, inflammatory cytokines, protein kinase, growth factors and receptors.

Turmeric produces neuro-protection by inhibiting acetylcholine and thereby increasing serotonin levels. Curcumin inhibits the activity of acetylcholinesterase but tetrahydrocurcumin inhibits the activity of acetylcholinesterase twice as well as Curcumin. Studies have shown that the neurological disorders such as depression are mainly caused by neurotransmitter hypo activity, due to not enough serotonin activity. Turmeric also provides neuro-protection in Alzheimer's disease in numerous ways i.e. inhibition of AB Peptide production, blocking the aggregation of peptides, reducing phosphorylation, reducing the levels of cholesterol, protecting the blood brain barrier, inhibition of acetylcholinesterase and decreasing inflammation and oxidative injury (Sharifi-Rad et al., 2020). Tetrahydrocurcumin has shown to be more effective than Curcumins in their anti-angiogenic activities. Because of these attributes the conclusion can be drawn that this formula can potentially open a promising healing window in the host that can accelerate the healing process.

### Turmeric/Piperine Synergy

Piperine is the main constituent and an alcoholic extract of the plant Piper nigrum, also known as black pepper. It has been found out to alleviate the symptoms of cognitive health (Wang et al., 2019). Piperine increases the bioavailability of Curcumin, and enhances it's the anti-inflammatory effect. (Chaiken, 2019). It has also been proven that Curcumin and piperine synergistically reduce pain (Boonrueng et al., 2022).

Clinical studies have shown proven efficacy of the combination of Curcumin and piperine in improving cognitive health (Boonrueng et al., 2022). Piperine is helpful in attenuating neuro inflammation thus promotes neurotransmission and cognition (Wang et al., 2019) and therefore it has been hypothesized that piperine can be used as a potential multi-targeting therapeutic option against certain neurodegenerative diseases such as Alzheimer's.

(Chonpathompikunlert, Wattanathorn and Muchimapura, 2010; Wang et al., 2019; 1, Sharma and An, 2023). Piperine reduces the force of H 2 O 2 related free radical synthesis by increasing the membrane potential of mitochondria, the main site of production of Reactive Oxygen Species and thereby minimizing the effects of oxidative stress (1, Sharma and An, 2023). The increase of membrane potential improves the life span of neuronal survival and help protect the brain from cerebral ischemia (1, Sharma and An, 2023). The anti-acetylcholinesterase activity and the anti-amyloid activity of piperine results in an anti-oxidative effect by maintaining the levels of antioxidant enzymes intact (Chonpathompikunlert, Wattanathorn and Muchimapura, 2010; 1, Sharma and An, 2023).

### Tetrahydrocurcuminoids

This formula uses Curcumin C-3 Reduct® by the Sabinsa Corporation as the proprietary source of turmeric containing tetrahydrocurcuminoids including: tetrahydrocurcumin, tetrahydrodesmethoxycurcumin and tetrahydrobisdesmethoxycurcumin obtained from the rhizomes of turmeric; Curcuma longa. Tetrahydrocurcuminoids are hydrogenated derivatives of Curcuminoids including Curcumin, desmethoxycurcumin and bisdemethoxycurcumin.

Curcumin C-3 Reduct® is more bioavailable than other Curcuminoids that are not modified. Tetrahydrocurcumin is more easily absorbed into the body than Curcumin when ingested orally. When compared to Curcumin, tetrahydrocurcuminoids are more pH stable in physiological conditions, photo-stable, tasteless, colorless, non-staining and are more potent antioxidants than ascorbic acid and grape seed extract.

The anti-oxidants present in this compound are cascading anti-oxidants. Curcumin C-3 Reduct® contains a patented extract of turmeric that has been standardized to 95% Tetrahydrocurcuminoids (THC's). The product is GRAS affirmed by the USDA. Curcumin C-3 Reduct® is a non-GMO verified, halal certified, Kosher certified, ISO Food Safety System 22000 approved and allergen free. In comparison to three other proprietary blends of 95% Curcuminoid containing turmeric products, Curcumin C-3 Reduct® by the Sabinsa Corporation stands out for its high-quality manufacture, patented, aesthetically acceptable nature, better dosage efficiency, certified and award-winning characteristics.

### Curcumin C-3 Reduct®

### Potential Biochemical and Metabolic Action

Curcumin produces its anti-oxidative and anti-inflammatory effects in various ways. The anti-oxidative effect is produced by scavenging radicals such as Nitric Oxide, Hydrogen peroxide and superoxide, by enhancing oxidative enzymes such as Superoxide Dismutase, CAT, GPX, OH-1 and thereby inhibit lipid peroxidation, increase glutathione by up regulation of GSH-transferase and their mRNAs, inhibit Reactive Oxygen Species producing enzymes such as COX, LOX, Xanthine Oxidase and scavenge peroxyl radicals (Sharifi-Rad et al., 2020). The anti-inflammatory effect is produced by the inhibition of proinflammatory transcription factors NEkB, AP-1, reduction of proinflammatory cytokines TNF-alpha, IL-1b, IL-2, IL-6, IL-8, MIP-1a, MCP-1, CRP, PGE2, down regulation of enzymes 5-Lipoxygenase, COX-2, COX-5, inhibition of nitrogen activated protein kinases and inhibition of pathways involved in Nitric Oxide synthase synthesis (Sharifi-Rad et al., 2020; Moudgil and Venkatesha, 2022). Studies show that Curcumin is able to inhibit the induction of COX-2 gene expression.

Curcumin inhibits the production of inflammatory cytokines and prostaglandins in activated microglia and astrocytes, inhibit the production of TNF-alpha, certain interleukins, macrophage-inflammatory protein, monocyte chemo-attractant protein in microglial and astrocyte cells and modulate several targets such as transcription factors, enzymes such as COX, LOX, NO and XO, inflammatory cytokines, protein kinase, growth factors and receptors. Studies have shown that the neurological disorders such as depression are mainly caused by neurotransmitter hypoactivity, due to not enough serotonin activity. Turmeric produces neuro-protection by inhibiting acetylcholine and thereby increasing serotonin levels. Turmeric also provides neuro-protection in Alzheimer's disease in numerous ways i.e. inhibition of AB Peptide production, blocking the aggregation of peptides, reducing phosphorylation, reducing the levels of cholesterol, protecting the blood brain barrier, inhibition of acetyl cholinesterase and decreasing inflammation and oxidative injury (Sharifi-Rad et al., 2020). Because of these attributes the conclusion can be drawn that the PetJuvenate®® formula can potentially open a promising healing window in the host, that can accelerate the healing process.

The patented “White Curcumin” C-3 Reduct® by the Sabinsa Corporation (used under license) is the formulation's proprietary source of Turmeric/Curcumin. A “Scientific Achievement Award” has been received by the Sabinsa Corporation for this product. According to the Nutrition Business Journal C-3 Reduct® contains a patented extract of turmeric that has been standardized to highly efficacious 95% Tetrahydrocurcuminoids. The product has received the Nutrition Business Award Journal Award for the Supplement Ingredient in 2008 and other awards such as the Functional Ingredient Marketing Award in 2012.

### Sunflower Lecithin

Sunflower Lecithin contains phosphatidylcholine and phosphatidylserine that has a huge bonus in promoting cognition and Sunflower Lecithin also acts as an emulsifier in the formula. Its ability to increase bioavailability is a major contributor to maximizing the efficacy of the formula. The anti-oxidant properties and cell repair benefits, stimulate memory and learning abilities. Sunflower Lecithin also aids in vitamin absorption, contains essential fatty acids and improves immune functions in the body. Sunflower Lecithin is one of the synergistic constituents powering the bioavailability of the Omega 3 Fatty acids present in the formula derived from the various ingredients. Omega-3 is proven to reduce cortisol levels in the body and studies show promising effects of in the treatment of depression and anxiety. It also performs in a similar fashion with other novel ingredients like CBD and more.

Lecithin's and Piperine's ability to make vital nutrients become exponentially more bioavailable to enhance brain function, in addition to the ability of the Astaxanthin and Manganese absorption ability that allows them to cross blood brain barrier and influence vital functions, all make up additional complimentary components of the PetJuvenate® formulation and accentuate the actual synergistic and really harmonizing mechanics created by the formulation.

In addition, other studies show that Lecithin also has properties to improve skin and coat quality due to the presence of the essential fatty acids, and the Flaxseed Meal, Keratin and Astaxanthin also produce skin/coat benefits. A definite added, useful and important health benefit of this formulation.

### Potential Biochemical and Metabolic Action

An alternative to Soybean Lecithin and a non-Genetically Modified product. It is high in palmitic acid and oleic acid. The major active components in sunflower Lecithin are phophatidylcholine, phophatidylethanolamine, phosphatidylinositol, phosphatidic acid, phosphotidylglycerol, triglycerides, fatty acids, carbohydrates and sphingolipids/phospholipids are accumulated in humans and animals in their vital organs and are important in maintaining cell membrane integrity, regulating cell to cell signaling, preventing neurological disease and act as a major structural component of cells (Malik and Tlustos, 2022). Phospholipids are considered as “Nootropic” for their ability to act as cognitive enhancers (Malik and Tlustos, 2022). They affect the metabolism of neuronal cells in the nervous system. They are used as supportives in acute to chronic nerve disorders such as memory loss, learning disorders, Alzheimer's and senile dementia. Sunflower Lecithin is used in improving memory as it releases Choline and acts as a precursor for acetylcholine synthesis.

Studies show that memory greatly improves with the supplementation of phophatidylcholine. It has also been found that increased levels of Lecithin in blood minimize ageing. Choline can also be produced by SAM supplementation via the SAM dependent trans methylation, which restores concentrations of Choline and acetylcholine (Shea, 2019). This proves that dietary Choline supplementation increases concentration of Choline in blood confirming the benefit of consuming food containing Choline to address multiple nerve disorders. To preserve the concentrations of cholinesterase the most preferred approach is to consume a single metabolite that leads to an increase in the cholinesterase enzyme concentration. Using dietary Choline sources is the best approach in this regard, as it will increase plasma Choline levels and inhibit cholinesterase. This will increase the release of acetylcholine in the brain. This is where this formula again shows its promise for neuro-supplementation and the phytomedical benefits.

An interesting side note. Lecithin supplements are used by some dog trainers, before and during training to improve cognition and learning behaviors (Puainta, 2023). Sunflower Lecithin is more expensive than Soy Lecithin. The formulators prefer sunflower Lecithin, for its superior quality, from extraction through processing, and also for its minimal post-consumption effects. Soy Lecithin is allergenic in some individuals, genetically engineered and may alter hormonal levels in the body. Whereas, Sunflower Lecithin is not extracted using harmful chemicals like ethanol, and it is the only type of Lecithin that is natural and chemical free. Sunflower Lecithin is processed by the method of cold pressing and therefore requires minimal alteration of the natural compounds, making it safer for consumption than Soy Lecithin. This was a qualifying reason this ingredient was selected by the formulator, and achieves yet another milestone in building a safe, natural, “Mind and Body Wellbeing” formulation.

### Boswellia Extract

Boswellia extract is also a renowned ancient Ayurveda medicine that has been used for its anti-inflammatory properties. It has been used to reduce lameness, localized pain and stiff gait thus, improving joint mobility. Recent clinical experimentation has confirmed Boswellia's ability to provide cartilage protection. https://pubmed.ncbi.nlm.nih.gov/18667054/ Studies also prove the effects of Boswellia extract on improved cognition as well as on boosted immunity to keep the immune mechanisms at equilibrium. This extract is used at present in therapy for conditions such as osteoarthritis, rheumatoid arthritis, cancer, inflammatory bowel disease, Parkinson's disease and asthma.

### Potential Biochemical and Metabolic Action

The extract of Boswellia serrata is gum resin which is also known as Indian Frankincense or Salai guggal, is an ancient medicine that has been used in the Ayurveda to treat chronic inflammatory diseases. The biologically active compound in Indian Frankincense is pentacyclic triterpenoids that comprises of a beta carboxyl moiety at C-24. The pentacyclic triterpenoids include Beta-Boswellic acid (BBA), Acetyl-11-keto-beta-Boswellic acid (AKBBA) and acetyl-beta-Boswellic acid (ABBA) (Siddiqui, 2011; Mehta, Dureja and Garg, 2016). Gum resin has anti-inflammatory, anti-arthritic and analgesia properties. The targeted pathways of Boswellic Acids to exert their actions include 5-LOX, Akt, COX-2, MAPK, NF-kB, Nrf2, STAT3, ERK1/2MMP9 and VEGF (Moudgil and Venkatesha, 2022). Studies have shown that Boswellic Acids in combination with Curcumin and ginger have proven results in the management of osteoarthritis. The beta configures Boswellic Acids inhibit 5-LOX resulting in an inhibition of leukotriene synthesis, which reduces the pain (Mehta, Dureja and Garg, 2016). Pentacyclic triterpenes uniquely inhibit both the 5-LOX pathway and human leukocyte elastase. AKBBA and BBA act in synergy to produce an anti-inflammatory activity in which AKBBA is the most potent inhibitor of the 5-LOX mechanism (Yu, Xiang and Zhang, 2020; Perez-Pinero, Munoz-Carrillo and Victoria-Montesinos, 2023).

Boswellic Acids inhibit the production of Th1 related cytokines and up regulate the production of Th2 related cytokines contributing to the anti-arthritic and anti-inflammatory effects (Moudgil and Venkatesha, 2022). This effect is used in the treatment of patients with joint pain and osteoarthritis (Perez-Pinero, Munoz-Carrillo and Victoria-Montesinos, 2023). The analgesic activity of Boswellia occurs by the activation of LOX pathway via an allosteric site. It has been found out that the efficacy of pain relief by Boswellia extracts is greater than the effect of pain relief by ibuprofen and glucosaminosulfate. The mode of action of Boswellic Acids is different to that of Non-Steroidal Anti-Inflammatory Drugs (NSAIDS) as Boswellic acids, as their major effects inhibit the 5-LOX pathway to reduce the production of leukotrienes and inhibit the activation of NF-kB and as their minor effect inhibit prostaglandin synthesis whereas NSAIDS mainly influence the COX pathway to reduce the production of PG synthesis. NSAIDS may cause a disturbance in the glycosaminoglycan synthesis but Boswellic Acids do not disturb this pathway and will help keep the cartilage intact . . . potentially safer compared to NSAIDS (Siddiqui, 2011). Boswellia Serrata extract decrease the glycosaminoglycan degradation. The BBA of Boswellia Serrata extract inhibit PGE synthase-1 inhibiting the production of PGE exerting the anti-inflammatory effect.

Boswellic Acids produce both a humoral response and cellular defense. In auto immune diseases there is an imbalance between cytokines and the T cells and there will be an abnormal antibody response. Boswellic Acids influence the immune system in several ways such as by the inhibition of proinflammatory cytokines, increased phagocytosis, deviated antibody response and the inhibition of the classical complement pathway (Moudgil and Venkatesha, 2022).

Inositol acetate is a potent anti-anxiety compound (Obistiolu et al., 2023). Increased levels of inositol acetate have shown to improve memory and cognition. KBA and AKBA inhibit p-glycoproteins in the brain and in the presence of inositol acetate in the Boswellia resin, prevent inflammation. Inositol acetate activates the TRPV1 channel and the TRPV3 channel. The activation of the TRPV3 channel promotes a Calcium influx. Calcium is a major stimulus for neurotransmission and signaling. Increased Calcium influx will enhance the hippocampus to transmit signals thus, improving memory. Boswellic Acids in combination with Curcumin and have proven results in the management of osteoarthritis, which boosts the synergistic use of these and other ingredients in this formula.

### Egg Shell Membrane (ESM)

Egg Shell Membrane provides nutrients that promote joint health and contain They are effective in reducing joint pain and increase joint function rapidly. ESM is being used therapeutically in promoting joint/bone health (to minimize the risks of Osteoporosis) and is being successfully tested to act as a semi-permeable membrane in wound healing.

Egg Shell Membrane is primarily made of fibrous proteins, such as Collagen types I, V, and X. It also contains Glycosaminoglycans (GAGs), like Dermatan Sulfate and Chondroitin Sulfate, as well as Hexosamines, like Glucosamine. Other ingredients include: Hyaluronic acid, Sialic acid, Desmosine, Isodesmosine, Ovotransferrin, Lysyl oxidase, Lysozyme, P—N-acetylglucosaminidase, and Ovocalyxin-36.

### Potential Biochemical and Metabolic Action

ESM is mainly composed of fibrous proteins such as collagen type 1, glycoaminoglycans such as deramatan sulfate and chondroitin sulfate, sulfated glycoproteins such as hexamines including glucosamines and other molecules such as hyaluronic acid and salicylic acid. ESM is considered a natural source of combined collagen, glucosamine, chondroitin and hyaluronic acid as a therapeutic for pain related conditions of joints and tissues. It is clinically proven that ESM supplementation significantly reduced pain in the joints (Ruff et al., 2009). Due to the versatility of ESM, it can be clinically modified to develop novel material for bone regeneration. ESM satisfies most criteria to act as a bone scaffold material due to its properties of bone regeneration, zero cytotoxicity and its anti-inflammatory properties. ESM has inherent properties as an adsorbent due to the presence of several functional groups on its surface such as —OH, —COOH, —SH, —NH2 and —CONH2 and help ESM to act as an anti-oxidant as well (DeVore and Long, 2007).

ESM is able to disrupt the occurrence of oxidative stress caused by H2O2 by suppressing the formation of H2O2 induced malondialdehyde and protein carboxyl by improving anti-oxidant enzyme synthesis and improving glutathione synthesis. ESM limits dysbiosis and produces anti-inflammatory action by inhibiting the production of inflammatory cytokine and by enhancing Caco-2 cell proliferation. These processes will improve gene expression of the related mediators, epithelial cell proliferation and anti-microbial peptides to exert an anti-inflammatory and an anti-microbial effect. ESM encourages osteoblast proliferation and angiogenesis by preventing epithelial migration. ESM accelerates wound healing by inhibiting CD11b− positive immune cells and by increasing the number of myofibroblast cells and proliferative cells.

### Green Oat Extract—Neuravena®

Dating back to the Medieval Era, green oat preparations were documented and shown to be used to treat brain disorders (Kennedy et al. 2017; 20(2); 135-151). Avena sativa has a wide range of health benefits that support vitality and is nutrient dense in iron, manganese and zinc. It is considered as a “nervine” for its ability to regulate the nervous system. The calming effects of Green Oat Extract reduces stress and improves relaxation thus, promotes cognitive functions by providing mental clarity, enhancing focus, memory and concentration. The anti-inflammatory and anti-oxidant properties of Green Oat Extract improves skin health, promotes skin regeneration and improves skin complexion.

### Potential Biochemical and Metabolic Action:

A non-GMO verified product. The benefits of Green Oat extract are mostly related to nervine processes and cognition. It has biologically active compounds such as avenacins, phenolic acids and polyphenols such as flavonoids and avenanthramides (Kennedy et al., 2020). Avenanthramides are useful in signal transduction in the brain by interacting with neurotransmitter receptors both directly and indirectly. Triterpenes also regulate nerve signaling by receptor interactions by inhibiting oxidation catalyzing enzymes, by hydrolyzing neurotransmitters or by controlling the glucocorticoid and estrogen systems.

Green Oat Extract has a strengthening and a balancing effect on brain and mind by demonstrating a dual activity portfolio on monoamine oxidase-B (MAO-B) and PDE4. PDE4 enzymes are responsible for functions related to cognition and mood. Green Oat Extract upregulates the monoamine neurotransmitter function by specifically inhibiting monoamine oxidase B and PDE4 (Ali Esmail Al-Snafi, 2015). The high levels of GABA in fermented oats reduce pain and improve anxiety and mood. A. sativa contains flavonoids and stilbene resveratrol that promote a cognitive function following a single dose Flavonoids and stilbene resveratrol increase blood flow to the cerebrum by increasing the synthesis of neurotropins and nitric oxide. Resveratrol is responsible in modulating inflammation and autoimmune diseases (Moudgil and Venkatesha, 2022). It has been found out that flavonoids delay the occurrence of neurodegenerative disorders and cognitive impairments. It is a well-known fact that the duration of diabetes is directly related to the level of cognition.

Another way by which A. sativa promotes cognition is by minimizing lipid peroxidation. The avenalin contained in A. sativa a legume-like protein, gluten and zein. These oligopeptides control hypoxic responses, increase oxygen carrying capacity and promote angiogenesis to minimize lipid peroxidation which ultimately provides protection to the brain against lactic acidosis and thereby promote cognitive health. A single dose of A. sativa demonstrates improved cognitions and therefore long-term supplementation is expected to modulate physiological responses to oxidative stress (Kennedy et al., 2020). A. sativa contains mucilage that drop glycemic levels and cholesterol responses (Jibril et al., 2023).

Used under License, Neuravena® is the science backed, Superior Green Oat Extract that is included in the formula. It contains a proprietary extract of Superior Green Oat that is high purity and stability thanks to the patented EFLA® HyperPure process used to process the extract. Neuravena® is superior in function when compared to other types of extracts of oats available in the market. Green oats are harvested when the seeds are still unripe. IFF Health (the manufacturer) has developed a selected green oat variety using a bioassay aiming at exerting these biofunctional effects. Safety and efficacy tests on animals have been conducted on Neuravena® and it is Halal certified, Kosher certified, non-irradiated and it contains no ingredient that might pose a risk of Bovine Spongiform Encephalopathy or Transmissible Spongiform Encephalopathy.

### Hyaluronic Acid

Hyaluronic Acid is naturally present in the synovial fluid and cartilage. it has been largely studied in the field of tissue engineering for its ability to regenerate cartilage by controlling inflammation. In vitro and in vivo studies have also shown the ability of Hyaluronic Acid to improve lubricate cartilage, improve cell adhesion and promote cell proliferation and thereby enhance deposition of extracellular matrix deposition and regeneration at cartilage. Hyaluronic Acid is able to identify the CD44 Hyaluronic Acid receptor to exert its biological effects on protecting cartilage and actively participating in tissue cartilage repair.

Hyaluronic Acid in the supplement provides higher concentrations of Hyaluronic Acid in synovial fluid and reduces concentrations of paraoxonase 1 in the synovial fluid. These improve mobility of joints by synthesizing more glycosaminoglycan by chondrocytes and synoviocytes. Hence, the cell proliferative properties of Hyaluronic Acid and the provision of viscoelasticity to tissues by Hyaluronic Acid make it an excellent ingredient to promote joint health.

### Potential Biochemical and Metabolic Action

A main component in the extracellular matrix of the brain and regulates the behaviors of cells in the central nervous system by regulating homeostasis. The functionality of the central nervous is disrupted when there is a loss of cell functional equilibrium by injury or disease. Hyaluronic Acid can influence tissue healing and cell signaling by producing pro-inflammatory mediators of low molecular weight hyaluronic acid, and inhibiting pro-inflammatory mediators of high molecular weight Hyaluronic Acid (Jensen, Holloway and Stabenfeldt, 2020; Ianconisi, Lunetti and Gallo, 2023).

Hyaluronic Acid increases Choline acetyl transferase expression in the medial septal nucleus and in the hippocampus. This effect of cholinergic septo-hippocampal innervation system supports hippocampal pyramidal neurons to promote synaptic transmission and promote cognition. There will be improved memory and reduced risks of estrogen deficiency induced cognitive impairment (Chen et al., 2023) specially in ovario-hysterectomized dogs and in Alzheimer's disease. Hyaluronic Acid is a main component of the synovial fluid in joints acting as a shock absorber and a lubricant. This property of Hyaluronic Acid is used in the treatment of osteoarthritis. The primary receptor of Hyaluronic Acid is CD44 (Salwowska, Bebenek and Zaio, 2016). It is a trans-membrane glycoprotein that is versatile. Hyaluronic Acid interacts with CD44 to internalize and degrade hyaluronan, to promote angiogenesis, to promote cell migration and influence the cell aggregation and adhesion to the extracellular matrix (Jensen, Holloway and Stabenfeldt, 2020).

Hyaluronic Acid inhibits macrophage production of PGE2 by down regulating NF-kB. High molecular weight Hyaluronic Acid reduces chemotaxis and cell migration to protect cells against free radicals, inhibit cell death of chondrocytes and stimulate proteoglycan synthesis and thereby produces a direct analgesic effect. By improving cartilage resilience, reducing inflammation and by preventing cartilage degradation Hyaluronic Acid increases the synthesis of cartilage matrix (Peck et al., 2021). During rapid wound repair it has been found out that increased levels of Hyaluronic Acid help retain the water content to create a receptive environment for wound healing (Juncan, Moisa and Santini, 2021; Ianconisi, Lunetti and Gallo, 2023).

### Manganese Chelate

Another ingredient of the formula is Manganese Chelate. This is the most absorbable oxidation state of Manganese which is why the formulator has included the Chelate form of Manganese in their formula. Manganese is an essential micronutrient and natural sources of Manganese include seeds, whole grains, nuts, legumes, beans, tea and leafy green vegetables. Manganese is required for the metabolism of amino acids, lipids and carbohydrates and it is the cofactor for many enzymes. Thus, Manganese assists in increased absorption of all other ingredients. The addition of Manganese Chelate is the “Catalyst” for all the ingredients working synergistically with their carefully calculated dosages to almost guarantee the trigger effect of chemical reactions in their respective channels and hence optimizing of the metabolic benefits of the formula.

### Potential Biochemical and Metabolic Action

There are several benefits exerted by Manganese in the body and they include supporting growth, metabolism, activating certain enzymes and supporting body functions such as immunity, reproduction and nerve regulation (Souza, 2023). Manganese also aids blood coagulation. In the nervous system Mn promotes neurotransmitter synthesis, metabolism and act as an anti-oxidant (Avila, Puntel and Aschner, 2013). Mn has versatile chemical properties and it has been found that Mn enables neuro-protection in mild cognitive impairment and in Alzheimer's disease. Mn can cross the blood brain barrier and the blood-cerebrospinal fluid barrier with the help of several carriers and oxidation states (Avila, Puntel and Aschner, 2013). The glutathione molecule contains Mn ions. The most abundant manganoprotein is glutathione (Avila, Puntel and Aschner, 2013). Glutathione is present in astrocytes to convert glutamate to glutamine (Avila, Puntel and Aschner, 2013). Insufficient levels of Mn can lead to exitotoxicity. This can ultimately result in generalized growth impairment, epilepsy, Down's syndrome, birth defects, reduced fertility and osteoporosis.

### Astaxanthin—AstaREAL®

Astaxanthin is a functional food with highly powerful anti-oxidant properties. Reputed to be multiple times more powerful than ascorbic acid in its anti-oxidant property, it is known as “The King of Caretonoids”. In times of intense oxidative stress ascorbic acid and alpha tocopherol become pro-oxidant and induce oxidative damage as opposed to its anticipated anti-oxidant effect but Astaxanthin is a rare anti-oxidant that will continue to scavenge free oxygen radicals even during intense oxidative damage without turning into a pro-oxidant. The natural structure of Astaxanthin being both lypophillic and hydrophillic with a transmembrane orientation enables it to act both in the intracellular compartment and extracellular compartment against oxidative attacks. It enhances the humoral response and promotes a cell mediated immune response as well.

Astaxanthin also inhibits the growth of mammary tumors and urinary bladder tumors, assists in managing obesity, hypertension and type-2 diabetes. AstaReal® also exerts anti-inflammatory effects by affecting the function of mitochondria to promote mitochondrial biogeny by activating the AMPK pathway and enhance ATP synthesis in mitochondria.

AstaReal® the proprietary Astaxanthin (used under license) in this formula contains natural patented Astaxanthin on which over seventy human clinical trials have been conducted to date. AstaReal® is considered as the world's most studied brand of Astaxanthin.

### Potential Biochemical and Metabolic Action

Astaxanthin is one of the most potent xanthophyll carotenoids found naturally. It shows superiority in absorbing free radicals when compared to alpha-tocopherol (Queen et al., 2024). Astaxanthin is considered as the naturally occurring ingredients with the highest oxygen radical absorption ability (Grimmig, Kim and Nash, 2017). Oxidative stress affects neuro degeneration, cognitive ageing, cognitive decline and diminished memory. Astaxanthin maintains cell integrity by improving gene expression and by boosting immunity. The ant-oxidant effect of Astaxanthin is exerted when it interacts with phosphor-inositide 3-kinase/protein kinase B pathway. This will neutralize the reactive oxygen species, destroy free radicals and manage lipid peroxidation. Astaxanthin has a different transport mechanism when compared to alpha-tocopherol, ascorbic acid or other carotenoids making it more efficacious in the brain functions.

Astaxanthin adopts all the lipoprotein densities and is able to cross the blood brain barrier. This helps Astaxanthin to act on both layers of the sarcolemma producing a greater efficacy. Prolonged oxidative stress increases free radicals and increases neuronal apoptosis. The weakening of the host anti-oxidant defense system by the reduced concentrations of SOD and HO-1 calms the activation of microglia, release pro-inflammatory cytokines and suppress excitotoxicity thus, improving cognition (Grimmig, Kim and Nash, 2017; Queen et al., 2024). It has been found out that the most realistic and practical approach to obtain Astaxanthin into the body is to use as a supplement and not through diet alone in order to maintain effective levels in the body. Astaxanthin is recognized as an approved dietary supplement since 1999 and is recognized as a GRAS material. The AstaReal used in PetJuvenate® formula is non-GMO with over 17 patents and the only brand of natural Astaxanthin with over 80 clinical studies and peer reviewed articles.

All of the above facts repetitively confirm that the formula is a true healer, with ingredients that can cross the “Blood Brain Barrier” and open a healing window to reduce host system stress. Reduced levels of pain accelerate healing. Each ingredient is clinically and in vitro proven effective (sometimes simultaneously) to relieve pain, or to minimize oxidative damage or improve gut microbiome function, and/or improve the cognitive state/mental stress levels. All of these compounds in the formula are formulated to work synergistically and effectively to reduce pain and promote healing. Hence, we have a novel and efficacious formula with considerable science behind it, and most suitable a for a “Mind and Body Wellbeing approach to supplementation.”

The inactive ingredients used in the formula are: Peanut Butter, Dried Potato Product, Brewer's Dried Yeast (low heat dried), Coconut Glycerin, Cane Molasses, Sunflower Oil, Sea Salt, Black Pepper Powder, Sorbic Acid (as found in Mountain Ash Berry), and Spray Dried Cheese Powder.

The observation is that compared to many supplements in the same segment, the formula concentrates on being “harsh chemical” free. A lack of excipients and harmful fillers in the formulation process, making it a very natural and presenting as more than 70% all botanical/herbal/plant-based formulation. This separates the formula for many supplements that use cheap, commercial, chemical derived preservatives or active ingredients that are non-human grade. This formula is indeed a human-grade and almost entirely a natural source product.

### The Initial Inflammatory Response

Inflammation is a normal defense mechanism in the body that identifies harmful agents and act against them to remove them from the body. Inflammation can be acute or chronic depending on its duration. Acute inflammation lasts from a few minutes to up to a few days. Chronic inflammation lasts from several days to several months or years. During acute inflammation event there will be vasodilation, increased blood flow, increased capillary vessel permeability and a diapedesis of neutrophils in the site of inflammation to act as a defense mechanism. Chronic inflammation is caused by the failure to eliminate the causative infectious agent from the body that causes an acute inflammation. An auto immune response by the body, the long-term exposure to low levels of a particular material that cannot be eliminated by the body properly by catabolism pathways, the continued production of free radicals produced by oxidative stress responses in the body that cannot be regulated by related mediators, and the imbalance in the inflammatory response in the body, can lead to a chronic inflammation.

At this time there will be infiltration of macrophages, plasma cells and lymphocytes through the capillaries at the site of inflammation. These chronic inflammatory cells will secrete inflammatory mediators to fight against the tissue damage and assist in the secondary tissue repair mechanism. Some of the other factors that contribute to low grade inflammation are the age, diet and obesity (Pahwa, Goyal and Jialal., 2023).

It has been found out that 56% of the dogs in the Unites States are obese and 51% dogs in the United Kingdom are obese (Kim K. Haddad, 2024). As a result of chronic low-grade inflammation, both human and pets are at risk of even the non-contagious diseases and disorders such as the nerve related cognitive disorders, arthritis and other joint related diseases, cardiovascular diseases, allergies, diabetes mellitus and cancer. Studies have shown that the prevalence of Canine Cognitive Disorder is estimated to be around 25% in dogs aged between 8 years and 12 years and around 70% in dogs above 15 years of age (Taylor et al., 2023). With globalization there is a possible imbalance between the oxidative and anti-oxidative chemicals in the body can lead to an increase of free radicals produced by various reactive oxygen species that can exacerbate negative health conditions in the body (Kelly et al., 2006). Inflammation is an integral process of the natural healing process of the body. Acute inflammation is the beneficiary response of the body to protect the body systems from various etiologies.

However, chronic inflammation may be detrimental and an alarming cause for concern. Can this formula be the host body's friend? A more kind and more gentle approach, allowing the Mind to have just as much a “say” in healing, as the body . . . if fact, “igniting” a more favorable healing overall . . . using both Mind and Body to control the healing processes. That is the solution that we believe and have demonstrated that this synergistic formulation can provide.

Healing involves a healthy mind and a healthy body. When the formula supplement is ingested the active ingredients in the formula will permeate the various metabolic pathways. The nervine agents in the Neuravena® Green Oat Extract and cognitive enhancing benefits of Sunflower Lecithin, Boswellia Extract, Manganese Chelate and AstaReal® Astaxanthin will increase nerve signaling in the brain and the entire nervous system. The anti-inflammatory mediators present in the Flaxseed Meal, Cynatine® FLX, Curcumin C-3 Reduct®, Hyaluronic Acid and the Boswellia will act on any ongoing chronic inflammation and provide a powerful analgesic effect in the joints, in the brain or elsewhere in the body. The anti-oxidant ingredients in the Flaxseed Meal, Egg Shell Membrane, Manganese Chelate and AstaReal® Astaxanthin will scavenge the free radicals' and further reduce chronic inflammation. The Keratin in the Cynatine® FLX, the Egg Shell Membrane, Boswellia, Curcumin and Hyaluronic Acid will help maintain healthy bones, cartilage and muscle tissues.

The formula is anticipated to exert its anti-inflammatory effect in several ways; the inhibition of the COX and the LOX pathways by either down regulating the COX and the LOX enzymes or by the inhibition of the COX and the LOX gene expression, release anti-inflammatory lipid mediators, release anti-inflammatory peptide mediators, inhibit the production of Nitric Oxide Synthase, increase phagocytosis or by the inhibition of the classical complement pathway. By releasing Omega 3 fatty acids, by increasing the synthesis of SOD or GSH, by reducing the concentrations of malondialdehyde, by scavenging the free radicals and by the inhibition of glycoproteins, the formula is expected to exert a harmonious anti-oxidant effect.

The nervine effect is produced by the formula by activating the neurotrophic factors, inhibiting inflammatory cytokines and lipid mediators in the glial cells and astrocytes, inhibiting acetylcholine concentrations to increase levels of serotonin, inhibiting the AB peptide production, blocking the aggregation of peptides, reducing phosphorylation, inhibiting acetylcholine-esterase, improving the mitochondrial membrane potential, induce SAM dependent trans methylation, inhibit glycoproteins and glucocorticoid systems, up regulate MAO-B and PDE4, minimize lipid peroxidation, improve acetylcholine transferase gene expression and by inhibiting the cholinergic septo hippocampal innervation system.

An analgesic effect will be produced by reducing chemotaxis, inhibiting the production of macrophages and by stimulating the synthesis of proteoglycans. the formula would also improve bone regeneration and wound healing by stimulating osteoblasts to release osteoclasts, up regulating the proliferation and activation of osteoclasts, increasing sulphur metabolism, up regulating chondrogenic markers, promoting Taurine-C1 synthesis, inhibiting CD11-b positive immune cells, increasing the number of myofibroblast cells and proliferative cells, interacting with receptors to reduce hyaluronan to promote angiogenesis and cell migration and by hydrating the cells sufficiently to accelerate cell growth. By turning these different biological pathways on concurrently and consecutively, the formula will be the “Multi-Pathway Switch” to act as the anti-inflammatory response to chronic inflammation while allowing the body's own initial immune response to be triggered by the formula, in contrast to a pharmaceutical approach . . . and then the “synergistic reaction” is activated and the desired result accomplished. Reduced System Stress=Increased Healing/Wellbeing.

The patent by Kelly et al. of 2006 reveals the possible synergistic ingredients that can be used in formulation with Keratin based products including hyaluronic acid, glucosamine, chondroitin sulfate, amino acids and several others which confirm the synergistic effect and safety of the formula. 80% of the active ingredients of the formula are 100% natural. Hence, a well-controlled nervous system, minimal mental and physical fatigue, improved mobility and a well-coordinated body system as a whole is the targeted goal for supplementation. The formula will be working in harmony with the initial inflammatory response to open a window for the body's natural processes to support the healing process.

### Safety Analysis of the Active Ingredients

Determined dosage of the formula:
Dogs of <11.3 kg (<25 lb.): ¼ chew 11.3 kg-22.7 kg (25-50 lb.): ½ chew 22.7 kg-34 kg (50-75 lb.): 1 chew>34 kg (>75 lb.): 2 chews

### 1. Stabilized Flaxseed Meal

Safety levels for small dogs and cats: 0.125 g/kg. Big dogs: 0.23 g/kg. Each chew consists of 400 mg of Stabilized Flaxseed Meal. Flaxseed levels are safe.

### 2. Cynatine® FLX

It has been proven safe in humans and as human food. In vitro and in vivo cytotoxicity studies report no toxic effects of extracted Keratin confirming its safety to use as a high value protein ingredient that supports normal body functions. No studies were found depicting its safety levels on dogs. One chew contains 150 mg of Cynatine® FLX. Efficacy levels: The factory recommendation is 150 mg to 200 mg of Cynatine® FLX for a dog weighing 70 lbs. Human trials have shown to be effective in 6 to 8 days and in dogs they predict Cynatine® FLX to show effects in 30 to 60 days.

### 3. Turmeric-Curcumin C-3 Reduct®

Safety levels: 50 mg to 250 mg of Curcumin 3 times a day. 3.14% of pure turmeric powder is Curcumin. Each chew consists of 50 mg of stabilized turmeric/Curcumin. The turmeric levels in the product are safe.

### 4. Sunflower Lecithin

Safety levels: 1000-10,000 mg/kg. Each chew consists of 80 mg of Sunflower Lecithin. The level of Sunflower Lecithin in the product is safe.

### 5. Boswellia Extract

Safety levels: 400 mg per 10 kg body weight once a day. Each chew contains 50 mg of Boswellia extract. Safe levels of Boswellia extract are present in the formula

### 6. Egg Shell Membrane

Safety levels: 13.5 mg/kg once a day. Each chew consists of 50 mg of Egg Shell Membrane. Safe levels of the Egg Shell Membranes have been achieved in the formula

### 7. Neuravena-Green Oat Extract®

Safety levels: 3 mg/kg per day. Each chew contains 50 mg. Safe levels present in the formula. Hyaluronic Acid Safety levels are up to 26 kg body weight: 27 mg of HA per day. More than 26 kg body weight: 54 mg of HA per day. Each chew contains 5 mg of HA. The level of HA in the formula is safe.

### 8. Manganese Chelate

Safety levels: 0.16 mg/kg/day with an absorption efficacy of 10%. Each chew contains 3 mg of Manganese Chelate. Safe amounts of Manganese Chelate are present in the formula

### 9. Astaxanthin—AstaReal®*

0.3 mg/kg/day of efficacy levels have been determined. No toxicity levels have been determined. Each of these active ingredients has no interaction with any of the other active ingredients in the formula.

### Benefits of the Formula

a. Stabilized Flaxseed Meal
b. Build joint resilience
c. Reduces inflammation
d. Improves mobility and reduce pain
e. Protects the joints
f. Improved gut microbiome health: Natural prebiotic. Increase the viability of Probiotics.
g. Promotes heart health
h. Lower risk of obesity, diabetes, malignancy
i. Improved cognition—Calming
j. Promotes wound healing

### 10. Cynatine® FLX*

a. Acts as a primary defense against oxidative damage
b. Promotes glutathione synthesis
c. Contains the building blocks needed to stimulates cartilage regeneration
d. Support healthy epithelia of skin
e. Promotes skeletal muscle functions
f. Reduces inflammation

### 11. Turmeric-Curcumin C-3 Reduct®

a. Comprises of Tetrahydrocurcuminoids-Powerful and 30% more bioavailable
b. Reduces production of free radicals
c. Reduces inflammation
d. Improves cognition: Enhanced effect/bioavailability by combining with Piperine

### 12. Sunflower Lecithin

a. Acts as an emulsifier
b. Improves skin and coat quality
c. Promotes mental health—Focus—Calming

### 13. Boswellia Extract

a. Reduces lameness, local pain and stiff gait
b. Protects cartilage
c. Regulates the nervous system
d. Overall pain reduction shown to appear in as little as 5 days.

### 14. Egg Shell Membrane

a. Reduces joint pain
b. Improves joint function rapidly

### 15. Neuravena®—Superior Green Oat Extract

a. Promotes calming effects, brain function and mood.
b. Improved memory, cognition
c. Promotes gut health
d. Improved skin condition
e. Increased energy levels
f. Regulate fat levels
g. Reduced risk of type 2 diabetes
h. Regulate blood pressure

### 16. Hyaluronic Acid

a. Improved joint mobility
b. Promotes rebuilding of joints
c. Supports cartilage regeneration
d. Increased synovial fluid

### 17. Manganese Chelate

a. Improved absorption of nutrients into the system
b. Improves bioavailability of all other ingredients

### 18. Astaxanthin-AstaReal®

a. Acts as an anti-oxidant
b. Improves cognition Anti-inflammatory effects
c. Inhibit the growth of certain types of tumors
d. Reduces the risk of certain chronic diseases such as obesity, hypertension and type 2 diabetes.

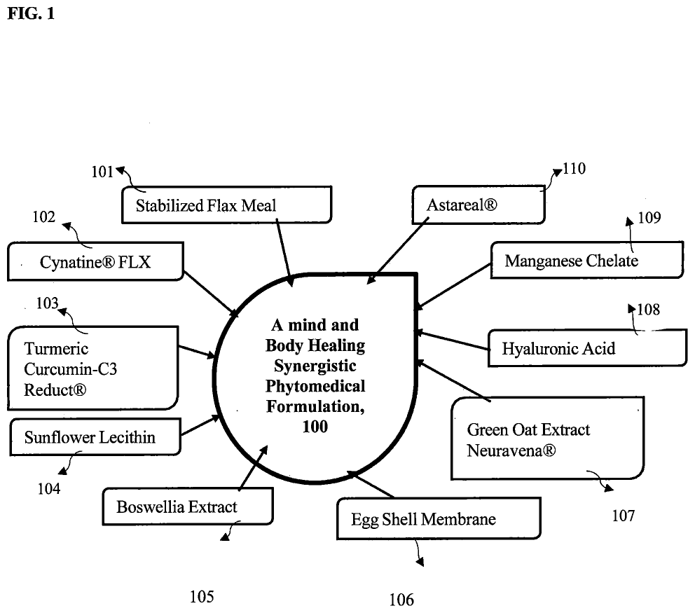

The primary active ingredients and their roles in the formulation are outlined in FIG. 1.

Stabilized Flax Meal (400 mg) according to FIG. 1 is rich in Omega-3 fatty acids and Lignans. Stabilized Flax Meal provides essential nutrients and bioactive compounds to promote overall health and enhance anti-inflammatory effects. Flaxseed is a natural anti-inflammatory and bolsters the immune system. It is also a natural prebiotic, which is essential for gut health and improved digestion.

In FIG. 1, Cynatine® FLX (150 mg) is derived from solubilized Keratin protein obtained from sheep's wool. Cynatine exhibits potent anti-inflammatory and antioxidant properties, supporting joint health and reducing symptoms of osteoarthritis. A source of sulfur-rich Cysteine helps contribute to the production of cartilage proteoglycans responsible for cushioning joints and may reduce synovial inflammation, fight free radicals and oxidative stress.

Sunflower Lecithin (80 mg): Serving as an emulsifier, Sunflower Lecithin enhances bioavailability and supports memory, learning, and immune functions, while also aiding in vitamin absorption as shown in FIG. 1. Essential for the well-being of all cells and is said to stimulate memory/learning abilities and enhance cognitive benefits. Contains, Choline, Phosphoric acid, Fatty Acids, Glycolipids and more.

### 19. Turmeric-Curcumin C-3 Reduct® (50 mg):

Derived from Curcumin C-3 Complex® by hydrogenation. The next generation, C-3Reduct® is a potent extract of cascading anti-oxidants called tetrahydrocurcuminoids (THC's). It supports healthy joint function, liver health, and cardiovascular wellness. Pharmacologically superior to standard Turmeric/Curcumin, it has increased chemical stability, increased antioxidant activity, much greater bioavailability. Additionally, its role in supporting the body's natural response to inflammation, and its antioxidant abilities, add another layer of active ingredients and their potential health benefits to our formulation.

Boswellia Extract (50 mg): A 100% natural ingredient containing AKBA (Acetyl-11-keto-beta-boswellic acid) and supports reduction of inflammation, soothe stiff/painful joints and muscles, and support healthy blood flow to synovial membrane, in addition to its antioxidant properties. Renowned for its anti-inflammatory properties in Ayurvedic medicine, Boswellia extract reduces pain and stiffness, improving joint mobility and providing cartilage protection.

Egg Shell Membrane (50 mg): Rich in Collagen, Glucosamine, and Hyaluronic Acid, Egg Shell Membrane promotes joint health, reduces pain, and increases joint function.

Neuravena®-Green Oat Extract (50 mg): Derived from Avena sativa, Green Oat Extract supports vitality, regulates the nervous system, reduces stress, and promotes cognitive functions, including mental clarity and concentration. Helps support enhanced optimization of neural resources and cognitive performance in tasks associated with executive functions, processing speed, attention and mood.

Hyaluronic Acid (5 mg): Helps lubricate and cushion joints, improves mobility and flexibility and can help with reduction of inflammation and easing of pain associated with normal wear and tear on joints. Enhancing synovial fluid concentrations/promoting joint mobility, Hyaluronic Acid synthesizes Glycosaminoglycan and provides viscoelasticity to tissues, supporting joint health.

Manganese Chelate (3 mg): Facilitating the metabolism of amino acids, lipids, and carbohydrates. Manganese Chelate acts as a co-factor for enzymes, increasing the absorption of other ingredients and enhancing their efficacy.

Astaxanthin-AstaReal® (2 mg): A functional food with antioxidant properties. AstaReal® supports muscle recovery and mitochondrial health, helping to maintain mobility and vitality. AstaReal® supports visual function, ocular hydration, and comfort. It also supports the canine immune system by helping to maintain healthy adaptive and innate immune functions. AstaReal® provides antioxidant support for aging dogs, helping to maintain mobility and healthy cardiovascular, cognitive, and visual function.

In addition to the active ingredients, the formulation contains a blend of inactive ingredients to enhance stability, palatability, and shelf life.

The synergistic botanical formulation described herein offers several advantages over conventional approaches to inflammation and nervous system dysfunction. By addressing both COX and LOX pathways, the formulation provides a dual-action approach to inhibit inflammation while promoting joint health and mobility. Furthermore, the incorporation of ingredients targeting the mental and cognitive state, such as Green Oat Extract and Sunflower Lecithin and others, offers a holistic approach to reducing anxiety and distress associated with chronic pain. The formulation's non-pharmaceutical approach utilizing natural ingredients represents an improved and accelerated method to improve quality of life for individuals suffering from inflammatory conditions and associated mental distress.

The formulation's unique blend of active ingredients, each chosen for their specific therapeutic properties, sets it apart from traditional treatments. Stabilized Flax Meal provides essential Omega-3 fatty acids and lignans, while soluble Keratin protein offers potent anti-inflammatory and antioxidant effects. Turmeric-Curcumin, a time-honored herbal remedy, contributes its own infamous anti-inflammatory and antioxidant benefits, complemented by the emulsifying and cognitive-enhancing properties of Sunflower Lecithin.

Boswellia Extract, Egg Shell Membrane, and Green Oat Extract bring centuries-old botanical wisdom to the formulation, providing targeted relief for joint discomfort, promoting joint health, and supporting cognitive function. Hyaluronic acid, Manganese Chelate, and Astaxanthin round out the formulation, each playing a crucial role in enhancing joint mobility, metabolic processes, and immune function, respectively.

Sunflower Lecithin also contains Phosphatidylserine. This highly effective nutrient has been used in several clinical studies for the support of cognitive function. Phosphatidylserine can also help decrease muscle soreness and reduce the number of stress hormones. This phospholipid is theorized to help the brain regulate mood, improves signaling, and some studies have shown improved memory and cognitive abilities. It supports memory and learning, supports executive function, supports mental flexibility, supports attention, supports number processing and calculation, supports cognitive health, supports acetylcholine release. It also supports monoamine oxidase B (MAO-B)*, supports brain-derived neurotrophic factor (BDNF), supports hippocampal IGF levels, supports the HPA axis, supports brain glucose metabolism, supports healthy EEG parameters, supports healthy mood and stress responses, supports positive affect, and supports healthy stress responses¹⁹.

In vitro, animal, and clinical studies have confirmed that Boswellia contains bioactive components that may enhance cognitive activity. Given the results of this study, Frankincense extract can reduce depression and anxiety in the LPS induced animal model. The frankincense extract also reduced IL-6 and TNF-α levels which supports the assumption that it has anti-inflammatory effects. Considering the fact that depression and anxiety are connected with inflammation, the present study suggests that treatment with frankincense extract can be beneficial to attenuate depression and anxiety symptoms associated with inflammation²⁰.

Beyond its coloring properties, Astaxanthin, a powerful carotenoid exhibits free radical scavenging, singlet oxygen quenching, and antioxidant activities which positively affect animal and human health with neuro protective qualities, with the ability to cross the blood-brain barrier.

These ingredients create a synergistic effect, amplifying their individual benefits and optimizing the formulation's overall efficacy. Furthermore, the formulation's emphasis on natural, and almost entirely plant-based ingredients aligns with modern trends towards holistic and sustainable healthcare solutions, appealing to a wide range of consumers seeking alternatives to conventional pharmaceuticals.

The synergistic botanical formulation represents a groundbreaking approach to therapeutic intervention, addressing both the physical and emotional aspects of inflammation and joint discomfort. By harnessing the power of nature's ingredients, this formulation offers a safer, more holistic, and more effective alternative to traditional treatments, paving the way for improved quality of life and well-being for individuals suffering from chronic pain and inflammation.

### BEST MODE OF CARRYING OUT THE INVENTION

The present invention provides a powerful anti-inflammatory for mind and body wellbeing supplement with the following natural ingredients.

In FIG. 1, the invention provides an anti-inflammatory COX/LOX pathways formulation 100 that reduces discomfort, muscle recovery, cartilage remodeling, but in addition addressing “Mental/Cognitive” state (anti-anxiety), comprising a combination of a stabilized flax meal 101 of between 250 mg and 500 mg; a Cynatine® FLX 102 of between 100 mg and 250 mg; a Curcumin C-3 Reduct® 103 of between 100 mg and 150 mg; a Sunflower Lecithin 104 of between 50 mg and 100 mg; a Boswellia extract 105 of between 20 mg and 70 mg; an Egg Shell Membrane 106 of between 20 mg and 70 mg; a Green Oat Extract-Neuravena® 107 of between 30 mg and 70 mg; a Hyaluronic Acid 108 of between of between 3 mg and 7 mg; Manganese Chelate 109 of between 2 mg and 5 mg, and an Astaxanthin-AstaReal® 110 of between 1 mg and 3 mg.

The invention uses Green Oat Extract-Neuravena® 107, Sunflower Lecithin 104, Flaxseed Meal 101 and Astaxanthin-AstaReal® 110 to create an approach of lessening the overall anxiety and distress caused by chronic pain and allowing the nervous system and gut microbiome to literally relax, be less stressed and let the anti-powerful and clinically tested inflammatory ingredients go to work. This opens the door to an improved and accelerated natural approach to utilizing mostly/herbs/plant-based ingredients to improve quality of life, with a non-pharmaceutical approach. Novel and ancient yet efficacious ingredients have been used in the product.

The main active ingredients per 4 g chew are.

| No | Ingredient | Quantity in mg | | Percentage |
|---|---|---|---|---|
| 1 | Stabilized Flax Meal | 400 | mg | 47.61% |
| 2 | Cynatine ® FLX | 150 | mg | 17.86% |
| 3 | Sunflower Lecithin | 80 | mg | 9.52% |
| 4 | Turmeric-Curcumin C-3 Reduct ® | 50 | mg | 5.95% |
| 5 | Boswellia Extract | 50 | mg | 5.95% |
| 6 | Egg Shell Membrane | 50 | mg | 5.95% |
| 7 | Green Oat Extract -Neuravena ® | 50 | mg | 5.95% |
| 8 | Hyaluronic Acid | 5 | mg | 0.60% |
| 9 | Manganese Chelate | 3 | mg | 0.36% |
| 10 | Astaxanthin -AstaReal ® | 2 | mg | 0.24% |

The inactive ingredients are: Peanut Butter, Dried Potato Products, Brewers Dried Yeast, Coconut Glycerin, Cane Molasses, Sunflower Oil, Sea Salt, Black Pepper Powder, Sorbic Acid (as found in Mountain Ash berry) and Spray Dried Cheese Powder.

Present that promotes good health. The stabilized Flaxseed Meal has a longer shelf life and therefore Flaxseed Meal 101 is a nutrient rich ingredient. It is considered as a “Superfood” for its many bioactive compounds more nutrients retain long term than its non-stabilized form. This is why the formulator uses the stabilized form of Flaxseed. The Flaxseed Meal 101 is the most bioactive form that absorbs the maximum number of into the system compared to Flaxseeds and Flaxseed Oil. The anti-inflammatory, anti-oxidant, anti-proliferative properties and the high fiber content of Flaxseeds provide exceptional health benefits.

Cynatine® FLX 102 is a very effective natural sourced ingredient that is derived from Sheep's Wool. Human joints are vulnerable to the effects of ageing due to the presence of oxidative stressors and frequent wear and tear of joints. To compensate the effects of various forces that deplete joints, scientists of New Zealand formulated a novel molecule comprising of a soluble form of Keratin protein that is bioactive, which comes to us as Cynatine® FLX 102. It is a “force multiplier” for the natural defense of the body. It consists of solubilized Keratin, a bioactive natural protein.

Randomized research shows the efficacy of Cynatine® FLX 102 on symptoms of osteoarthritis by reducing pain scores by twice as compared to placebo and shows the same efficacy in reducing stiffness. Solubilized Keratin rebuilds vital joint tissues with essential bioactive high sulphur precursors. The anti-inflammatory properties and the anti-oxidant properties contribute to promote joint health. When Cynatine® FLX 102 is ingested and metabolized, there is an increased production of superoxide dismutase and glutathione—a major anti-oxidant in the body. The increased levels of glutathione dependent peroxidase help as the primary defense against oxidative damage. The anti-oxidant property of solubilized Keratin increases mobility of joints by reducing the damage in joints caused by free radicals and by reducing pain in the joints.

Turmeric-Curcumin C-3 Reduct® 103 is an ancient herbal medicine that has been used through many generations as an anti-inflammatory and an anti-oxidant ingredient. In most of the South Asian countries' turmeric is used as a daily spice in food for its proven health benefits.

Turmeric-Curcumin C-3 Reduct® 103 regulates proinflammatory enzymes Cyclooxygenase (COX), Lipooxygenases (LOX)) and regulates the inflammatory transcription factors Nuclear Factor kappa B (NF-kB) and Signal Transducer and Activator of Transcription 3 (STAT3) thus, producing an anti-inflammatory effect in the whole-body system.

Sunflower Lecithin 104 acts as an emulsifier in the formulation. Its ability to increase bioavailability is a strong suit to maximize efficacy of the formula. The anti-oxidant properties and the ability of cell repair stimulate memory and learning abilities. Sunflower Lecithin also aids in vitamin absorption, contains essential fatty acids and improves immune functions in the body. Studies show that Lecithin also has properties to improve skin coat quality due to the presence of the essential fatty acids

Boswellia extract 105 is also a renowned ancient Ayurveda medicine that has been used for its anti-inflammatory properties. Also, it is known to reduce lameness, local pain and stiff gait thus, improving joint mobility. Recent clinical experimentation has also confirmed Boswellia's ability to provide cartilage protection.

Egg Shell Membranes 106 contain collagen, glucosamine and hyaluronic acid. Egg shell membranes 106 provide nutrients that promote joint health. They are effective in reducing joint pain and increase joint function rapidly.

Green Oat Extract 107 from the uppers of Avena sativa has a wide range of health benefits that support vitality and is nutrient dense in iron, manganese and zinc. It is considered as a “nervine” for its ability to regulate the nervous system. The calming effects of Green Oat Extract reduces stress and improves relaxation thus, promotes cognitive functions by providing mental clarity, enhancing focus, mood, memory and concentration. The anti-inflammatory and anti-oxidant properties of this extract improves skin health, promotes skin regeneration and improves skin complexion.

Hyaluronic Acid 108 in the supplement provides higher concentrations of Hyaluronic Acid in synovial fluid and reduces concentrations of paraoxonase 1 in the synovial fluid. These improve mobility of joints by synthesizing more glycosaminoglycan by chondrocytes and synoviocytes. Hence, the cell proliferative properties of Hyaluronic Acid and the provision of viscoelasticity to tissues by Hyaluronic Acid 108 make it an exceptional ingredient to maintain joint health.

Manganese Chelate 109 is the most absorbable oxidation state of Manganese which is why the formulator has included the chelated form of Manganese in their formula. Manganese is required for the metabolism of amino acids, lipids and carbohydrates and it is the co-factor for many enzymes. Thus, Manganese increases absorption of all other ingredients in formula giving it a better real-life effect.

Astaxanthin—AstaReal® 110 is a functional food with anti-oxidant properties. It also enhances the humoral response and promotes a cell mediated immune response as well. Astaxanthin 110 also inhibits the growth of mammary tumors, urinary bladder tumors and assists in managing obesity, hypertension and type 2 diabetes.

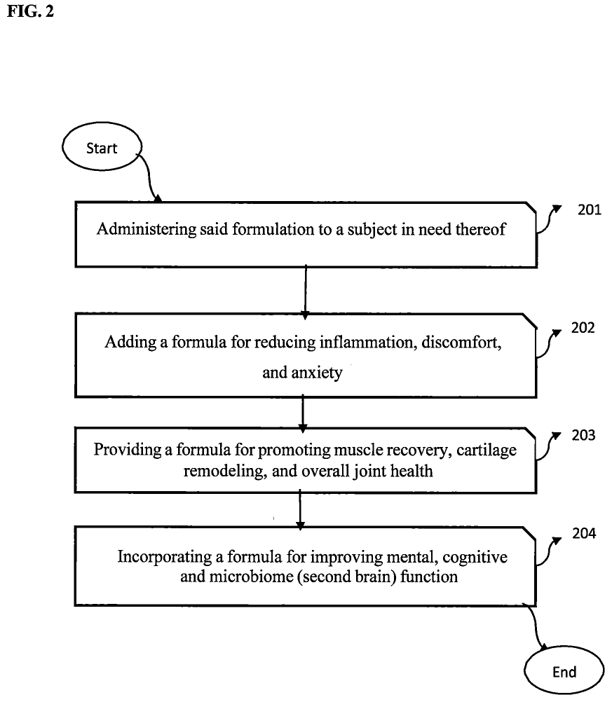

Turning to FIG. 2, the invention provides a novel method of formulating a synergistic phytomedical for therapeutic intervention, comprising steps of:
a. administering said formulation to a subject in need thereof at step 201;
b. adding a formula for reducing inflammation, discomfort, and anxiety at step 202;
c. providing a formula for promoting muscle recovery, cartilage remodeling, and overall joint health at step 203, and
d. incorporating a formula for improving mental and cognitive functions at step 204.

The said method further includes specific dual approaches to reduce pain and inflammation in dogs designed to address the anti-inflammation support and the equally important stressed mental state caused by chronic pain and causing a slower healing process.

The formulation further includes adding a composition that is formulated for fighting the mental stress caused by injury (pain and inflammation); incorporating a formula designed for improving mental state and mood (wellbeing) as a precursor and equal partner in faster, more targeted healing (mind and body wellbeing) and incorporating a synergistic formula for attacking inflamed joints/tissues and gut microbiome (second brain) and all with a non-pharmaceutical, natural botanically derived delivery vehicle.

### Advantages

Some merits of the present invention include:
a. provision of a multi-channel oriented, synergistic botanical healing formulation, addressing both Mind and Body wellbeing;
b. reduces anxiety, calm the mind, reduce the stress and consequently, the negative effects produced. Then once the host system is mentally primed and most receptive, opening the door for the other anti-inflammatory ingredients to activate faster and in a more targeted and efficient fashion, and
c. provision of synergistic botanical interactions is of vital importance in phytomedicines, to explain difficulties in always isolating a single active ingredient, and explain the efficacy of apparently low doses of active constituents in an herbal product.

The invention has combined known ingredients in a not previously introduced format to have a new and specific dual approach to pain and inflammation in dogs. It has addressed the anti-inflammation support and the equally important stressed mental state caused by chronic pain and causes a slower healing process. The invention is designed to fight the mental stress caused by injury (pain and inflammation), improving mental state and mood (wellbeing) as a precursor and equal partner in faster, more targeted healing while incorporating a synergistic attack on inflamed joints/tissues and gut microbiome and all with a non-pharmaceutical, botanic delivery channel.

While the invention has been described with reference to numerous specific sensors, one of ordinary knowledge in the field will recognize that the invention can be embodied in other specific forms without departing from the spirit of the invention. Specific operations may not be performed in one continuous series of operations, and different specific operations may be performed in different embodiments. Furthermore, the processes or methods could be implemented using several sub-processes, or as part of a larger macro process. Thus, one of ordinary knowledge in the field would understand that the invention is not to be limited by the foregoing illustrative details, but rather is to be defined by the appended claims.

### INDUSTRIAL APPLICATION

The present invention applies to pharmacology. It introduces a mind and body wellbeing phytomedical formulation. It comprises a synergistic botanical formulation with a dual approach to inhibit Cyclooxygenase (COX) and Lipoxygenase (LOX) enzymes while enhancing nervine function for therapeutic intervention encompasses pharmaceutical compositions and methods for treating inflammatory conditions and neurological and gut microbiome health. It provides a synergistic botanical formulation described herein has wide-ranging industrial applications in the pharmaceutical and nutraceutical sectors. It can be formulated into various dosage forms, including tablets, capsules, liquid extracts, powders and topical preparations, catering to diverse patient populations and therapeutic needs. The invention presents a formula that provides a whole or partially purified extract of a plant offers advantages over a single isolated ingredient. The synergistic interactions of constituents within a total extract of a single herb, as well as between different herbs in a formulation.

### Abbreviations

1. ALA Alpha Linolenic Acid
2. LOX—Liopxygenase
3. COX—Cycloxygenase
4. LT Leukotriene
5. BBA Beta Bosweillic Acid
6. AKBBA Acetyl-11-keto-beta-boswellic acid
7. GRAS Generally Recognized As Safe
8. LDL Low Density Lipoprotein
9. SOD Superoxide Dismutase
10. CAT Catalase
11. GPX Glutathione peroxidase
12. OH-1 Heme oxygenase
13. GSH—transferase Glutathione Transferase
14. NFkB Nuclear Factor kappa B
15. AP-1 Activator protein-1
16. TNF-alpha Tumour Necrosis Factor alpha
17. IL—Interleukin
18. ATP Adenosine Triphosphate
19. MIP-1a Macrophage Inflammatory Protein-1 alpha
20. MCP-1 Monocyte Chemoattractant Protein-1
21. CRP C-Reactive Protein
22. PGE Prostaglandin E
23. SAM S-Adenosyl Methionine
24. MAPK Mitogen Activated Protein Kinase
25. Nrf2 Nuclear Factor Erythroid 2-related Factor-2
26. STAT3 Signal Transducer and Activator of Transcription 3
27. ERK1/2MMP9 Extracellular Signal—regulated Kinase 1/2Matrix metallopeptidase 9
28. VEGF Vascular Endothelial Growth Factor
29. NSAIDS Non-Steroidal Anti-Inflammatory Drugs
30. TRPV1 channel Transient Receptor Potential Vanilloid subtype 1
31. TRPV3 channel Transient Receptor Potential Vanilloid subtype 3
32. ESM Egg Shell Membrane
33. H₂O₂ Hydrogen peroxide
34. Caco-2 Cancer coli
35. CD11b Cluster of Differentiation Molecule 11b
36. MAO-B Monoamine oxidase-B
37. PDE4 Prostaglandin E4
38. GABA Gamma Aminobutyric Acid
39. GMO Genetically Modified Organism
40. USDA United States Department of Agriculture

### REFERENCES

1. Gholami Z, Akhlaghi M. The effect of flaxseed on physical and mental fatigue in children and adolescents with overweight/obesity: a randomized controlled trial. Br J Nutr. 2021 Jul. 14; 126(1):151-159. doi: 10.1017/S0007114520003888. Epub 2020 Oct. 8. PMID: 33028427. https://mosscenterforintegrativemedicine.com/blog/2018/12/1/flaxseed-powder-a-powerful-regulator-of-cortisol
2. Al-Madhagy, S. et al. (2023) “A comprehensive review of the health benefits of Flaxseed oil in relation to its chemical composition and comparison with other Omega-3-rich oils,” European Journal of Medical Research, 28(1), p. 240. Available at: https://doi.org/10.1186/s40001-023-01203-6.
3. Giteru, S. G., Ramsey, D. H., Hou, Y., Cong, L., Mohan, A., & Bekhit, A. E.-D. A. (2023). Wool Keratin as a novel alternative protein: A comprehensive review of extraction, purification, nutrition, safety, and food applications. Comprehensive Reviews in Food Science and Food Safety, 22, 643-687. https://doi.org/10.1111/1541-4337.13087
4. Sharifi-Rad J, Rayess Y E, Rizk A A, Sadaka C, Zgheib R, Zam W, Sestito S, Rapposelli S, Neffe-Skocińska K, Zielińska D, Salehi B, Setzer W N, Dosoky N S, Taheri Y, El Beyrouthy M, Martorell M, Ostrander E A, Suleria H A R, Cho W C, Maroyi A, Martins N. Turmeric and Its Major Compound Curcumin on Health: Bioactive Effects and Safety Profiles for Food, Pharmaceutical, Biotechnological and Medicinal Applications. Front Pharmacol. 2020 Sep. 15; 11:01021. doi: 10.3389/fphar.2020.01021. PMID: 33041781; PMCID: PMC7522354.
5. https://link.springer.com/article/10.1007/s42250-022-00410-8
6. Malik M, Tlustos P. Nootropics as Cognitive Enhancers: Types, Dosage and Side Effects of Smart Drugs. Nutrients. 2022 Aug. 17; 14(16):3367. doi: 10.3390/nu14163367. PMID: 36014874; PMCID: PMC9415189.
7. Thomas B Shea. Choline and phosphatidylcholine may maintain cognitive performance by multiple mechanisms. The American Journal of Clinical Nutrition, 2019; 110(6):1268-1269. doi:10.1093/ajcn/ngz244.
8. Rajabian A, Sadeghnia H, Fanoudi S, Hosseini A. Genus Boswellia as a new candidate for neurodegenerative disorders. Iran J Basic Med Sci. 2020 March; 23(3):277-286. doi:10.22038/IJBMS.2020.35288.8419. PMID: 32440312; PMCID: PMC7229515.
9. Kennedy D O, Bonnländer B, Lang S C, Pischel I, Forster J, Khan J, Jackson P A, Wightman E L. Acute and Chronic Effects of Green Oat (Avena sativa) Extract on Cognitive Function and Mood during a Laboratory Stressor in Healthy Adults: A Randomized, Double-Blind, Placebo-Controlled Study in Healthy Humans. Nutrients. 2020 May 29; 12(6):1598. doi: 10.3390/nu12061598. PMID: 32485993; PMCID: PMC7352613.
10. Al-Snafi, Ali. (2015). The nutritional and therapeutic importance of Avena sativa—An Overview. International Journal of Phytotherapy. 5. 48-56.
11. Tijani Jibril, Aliyu & Arero, Amanuel & Kankam, Samuel & Fuseini, Miftau. (2022). Effect of Avena sativa (Oats) on cognitive function: A systematic review of randomized controlled trials. Clinical Nutrition ESPEN. 53. 10.1016/j.clnesp.2022.12.011.
12. Jensen G, Holloway J L, Stabenfeldt S E. Hyaluronic Acid Biomaterials for Central Nervous System Regenerative Medicine. Cells. 2020 Sep. 17; 9(9):2113. doi: 10.3390/cells9092113. PMID: 32957463; PMCID: PMC7565873.
13. Chen M-H, Lin H-C, Chao T, Lee V S-Y, Hou C-L, Wang T-J, Chen J-R. Hyaluronic Acid Conjugated with 17β-Estradiol Effectively Alleviates Estropause-Induced Cognitive Deficits in Rats. International Journal of Molecular Sciences. 2023; 24(21):15569. https://doi.org/10.3390/ijms242115569
14. Horning K J, Caito S W, Tipps K G, Bowman A B, Aschner M. Manganese Is Essential for Neuronal Health. Annu Rev Nutr. 2015; 35:71-108. doi: 10.1146/annurev-nutr-071714-034419. Epub 2015 May 13. PMID: 25974698; PMCID: PMC6525788.
15. Queen C J J, Sparks S A, Marchant D C, McNaughton L R. The Effects of Astaxanthin on Cognitive Function and Neurodegeneration in Humans: A Critical Review. Nutrients. 2024 Mar. 14; 16(6):826. doi: 10.3390/nu16060826. PMID: 38542737; PMCID: PMC10975052.
16. Obi tioiu D, Hulea A, Cocan I, Alexa E, Negrea M, Popescu I, Herman V, Imbrea I M, Heghedus-Mindru G, Suleiman M A, Radulov I, Imbrea F. Boswellia Essential Oil: Natural Antioxidant as an Effective Antimicrobial and Anti-Inflammatory Agent. Antioxidants (Basel). 2023 Sep. 27; 12(10):1807. doi: 10.3390/antiox12101807. PMID: 37891886; PMCID: PMC10603989.
17. Pérez-Piñero S, Muñoz-Carrillo J C, Victoria-Montesinos D, Garcia-Muñoz A M, Andreu-Caravaca L, Gómez M, Schölzel M, García-Guillén A I, López-Román F J. Efficacy of Boswellia serrata Extract and/or an Omega-3-Based Product for Improving Pain and Function in People Older Than 40 Years with Persistent Knee Pain: A Randomized Double-Blind Controlled Clinical Trial. Nutrients. 2023; 15(17):3848. https://doi.org/10.3390/nu15173848
18. Majeed M, Majeed S, Narayanan N K, Nagabhushanam K. A pilot, randomized, double-blind, placebo-controlled trial to assess the safety and efficacy of a novel Boswellia Serrata extract in the management of osteoarthritis of the knee. Phytotherapy Research. 2019; 33: 1457-1468. https://doi.org/10.1002/ptr.6338
19. Mehta, M., Dureja, H., & Garg, M. (2016). Development and optimization of Boswellic Acid-loaded proniosomal gel Drug Delivery, 23(8), 3072-3081. https://doi.org/10.3109/10717544.2016.1149744
20. Yu, G. et al. (2020) “Effectiveness of Boswellia and Boswellia extract for osteoarthritis patients: a systematic review and meta-analysis,” BMC Complementary Medicine and Therapies, 20(1), p. 225. Available at: https://doi.org/10.1186/s12906-020-02985-6.
21. Moudgil, Kamal & Venkatesha, Shivaprasad. (2022). The Anti-Inflammatory and Immunomodulatory Activities of Natural Products to Control Autoimmune Inflammation. International Journal of Molecular Sciences. 24. 95. 10.3390/ijms24010095.
22. Jalili C, Salahshoor M R, Moradi S, Pourmotabbed A, Motaghi M. The therapeutic effect of the aqueous extract of Boswellia serrata on the learning deficit in kindled rats. Int J Prev Med. 2014 May; 5(5):563-8. PMID: 24932387; PMCID: PMC4050676.
23. Grimmig B, Kim S H, Nash K, Bickford P C, Douglas Shytle R. Neuroprotective mechanisms of Astaxanthin: a potential therapeutic role in preserving cognitive function in age and neurodegeneration. Geroscience. 2017 February; 39(1):19-32. doi: 10.1007/s11357-017-9958-x. Epub 2017 Feb. 13. PMID: 28299644; PMCID: PMC5352583.
24. Marreilha Dos Santos A P, Andrade V, Aschner M. Neuroprotective and Therapeutic Strategies for Manganese-Induced Neurotoxicity. Clin Pharmacol Transl Med. 2017; 1(2):54-62. Epub 2017 May 26. PMID: 30854510; PMCID: PMC6402347.
25. https://www.news-medical.net/news/20231025/Boosting-brain-health-with-iron-and-manganese-A-dietary-defense-against-cognitive-decline.aspx
26. Avila, Daiana & Puntel, Robson. (2013). Manganese in Health and Disease. Metal ions in life sciences. 13. 199-227. 10.1007/978-94-007-7500-8_7.
27. Ruff K J, DeVore D P, Leu M D, Robinson M A. Eggshell membrane: a possible new natural therapeutic for joint and connective tissue disorders. Results from two open-label human clinical studies. Clin Interv Aging. 2009; 4:235-40. doi: 10.2147/cia.s5797. Epub 2009 Jun. 9. PMID: 19554094; PMCID: PMC2697588.
28. https://patents.google.com/patent/US8580315B2/en
29. Juncan A M, Moisa D G, Santini A, Morgovan C, Rus L L, Vonica-incu A L, Loghin F. Advantages of Hyaluronic Acid and Its Combination with Other Bioactive Ingredients in Cosmeceuticals. Molecules. 2021 Jul. 22; 26(15):4429. doi: 10.3390/molecules26154429. PMID: 34361586; PMCID: PMC8347214.
30. Iaconisi G N, Lunetti P, Gallo N, Cappello A R, Fiermonte G, Dolce V, Capobianco L. Hyaluronic Acid: A Powerful Biomolecule with Wide-Ranging Applications-A Comprehensive Review. Int J Mol Sci. 2023 Jun. 18; 24(12):10296. doi: 10.3390/ijms241210296. PMID: 37373443; PMCID: PMC10299688.
31. Salwowska, Natalia & Bebenek, Katarzyna & Żądło, Dominika & Wcislo—Dziadecka, Dominika. (2016). Physiochemical properties and application of hyaluronic acid: A systematic review. Journal of cosmetic dermatology. 15. 10.1111/jocd.12237.
32. Peck J, Slovek A, Miro P, Vij N, Traube B, Lee C, Berger A A, Kassem H, Kaye A D, Sherman W F, Abd-Elsayed A. A Comprehensive Review of Viscosupplementation in Osteoarthritis of the Knee. Orthop Rev (Pavia). 2021 Jul. 10; 13(2):25549. doi: 10.52965/001c.25549. PMID: 34745480; PMCID: PMC8567800.
33. Siddiqui M Z. Boswellia serrata, a potential anti-inflammatory agent: an overview. Indian J Pharm Sci. 2011 May; 73(3):255-61. doi: 10.4103/0250-474X.93507. PMID: 22457547; PMCID: PMC3309643.
34. Elizabeth Ignowski, Aimee N. Winter, Nathan Duval, Holly Fleming, Tyler Wallace, Evan Manning, Lilia Koza, Kendra Huber, Natalie J. Serkova, Daniel A. Linseman, The cysteine-rich whey protein supplement, Immunocal®, preserves brain glutathione and improves cognitive, motor, and histopathological indices of traumatic brain injury in a mouse model of controlled cortical impact, Free Radical Biology and Medicine, 2018, 124: 328-341 doi:10.1016/j.freeradbiomed.2018.06.026.
35. https://www.roxlor.com/cynatineflx.php
36. https://patents.google.com/patent/US7732574B2/en
37. Galluccio F, Barskova T, Cerinic M M. Short-term effect of the combination of hyaluronic acid, chondroitin sulfate, and Keratin matrix on early symptomatic knee osteoarthritis. Eur J Rheumatol. 2015 September; 2(3):106-108. doi: 10.5152/eurjrheum.2015.0019. Epub 2015 Sep. 1. PMID: 27708941; PMCID: PMC5047250.
38. Beer, Christina & Wood, Simon & Veghte, Robert. (2013). A Randomized, Double-Blind, Placebo-Controlled Clinical Trial to Investigate the Effect of Cynatine® FLX on Symptoms of Osteoarthritis. Journal of dietary supplements. 10. 10.3109/19390211.2013.822449.
39. https://patents.google.com/patent/WO2006099309A2/en
40. Eliza Ranjit, Stephen Hamlet, Roy George, Ajay Sharma, Robert M. Love. Biofunctional approaches of wool-based Keratin for tissue engineering. Journal of Science: Advanced Materials and Devices. 7(1)2022:100398. ISSN 2468-2179. doi:10.1016/j.jsamd.2021.10.001.
41. Wu Y-L, Lin C-W, Cheng N-C, Yang K-C, Yu J. 2017. Modulation of Keratin in adhesion, proliferation, adipogenic, and osteogenic differentiation of porcine adipose-derived stem cells. J Biomed Mater Res Part B 2017: 105B: 180-192.
42. Gera M, Sharma N, Ghosh M, Huynh D L, Lee S J, Min T, Kwon T, Jeong D K. Nano formulations of Curcumin: an emerging paradigm for improved remedial application. Oncotarget. 2017 Jul. 11; 8(39):66680-66698. doi: 10.18632/oncotarget.19164. PMID: 29029547; PMCID: PMC5630447.
43. https://puainta.com/blogs/care-healthy-living/Lecithin-benefits
44. Janakiram N B, Valerio M S, Goldman S M, Dearth C L. The Role of the Inflammatory Response in Mediating Functional Recovery Following Composite Tissue Injuries. Int J Mol Sci. 2021 Dec. 17; 22(24):13552. doi: 10.3390/ijms222413552. PMID: 34948349; PMCID: PMC8705789.
45. Pahwa R, Goyal A, Jialal I. Chronic Inflammation. [Updated 2023 Aug. 7]. In: StatPearls [Internet]. Treasure Island (FL): StatPearls Publishing; 2024 January-. Available from: https://www.ncbi.nlm.nih.gov/books/NBK493173/
46. Haddad K K. How Successful Are Veterinary Weight Management Plans for Canine Patients Experiencing Poor Welfare Due to Being Overweight and Obese? Animals. 2024; 14(5):740. https://doi.org/10.3390/ani14050740
47. Tracey L. Taylor, Eduardo J. Fernandez, Kimberley N. Handley, Susan J. Hazel. Non-pharmacological interventions for the treatment of canine cognitive dysfunction: A scoping review, Applied Animal Behavior Science, 2023(269), 106097, ISSN 0168-1591
48. Wang C, Cai Z, Wang W, Wei M, Kou D, Li T, Yang Z, Guo H, Le W, Li S. Piperine attenuates cognitive impairment in an experimental mouse model of sporadic Alzheimer's disease. J Nutr Biochem. 2019 August; 70:147-155. doi: 10.1016/j.jnutbio.2019.05.009. Epub 2019 May 24. PMID: 31207354.
49. Sharma H, Sharma N, An S S A. Black Pepper (Piper nigrum) Alleviates Oxidative Stress, Exerts Potential Anti-Glycation and Anti-AChE Activity: A Multitargeting Neuroprotective Agent against Neurodegenerative Diseases. Antioxidants (Basel). 2023 May 12; 12(5):1089. doi: 10.3390/antiox12051089. PMID: 37237954; PMCID: PMC10215410.
50. Chonpathompikunlert P, Wattanathorn J, Muchimapura S. Piperine, the main alkaloid of Thai black pepper, protects against neurodegeneration and cognitive impairment in animal model of cognitive deficit like condition of Alzheimer's disease. Food Chem Toxicol. 2010 March; 48(3):798-802. doi: 10.1016/j.fct.2009.12.009. Epub 2009 Dec. 23. PMID: 20034530.
51. Derosa G, Maffioli P, Sahebkar A. Piperine and Its Role in Chronic Diseases. Adv Exp Med Biol. 2016; 928:173-184. doi: 10.1007/978-3-319-41334-1_8. PMID: 27671817.
52. https://www.umassmed.edu/nutrition/blog/blog-posts/2019/6/using-black-pepper-to-enhance-the-anti-inflammatory-effects-of-turmeric/
53. Nowak W, Jeziorek M. The Role of Flaxseed in Improving Human Health. Healthcare (Basel). 2023 Jan. 30; 11(3):395. doi: 10.3390/healthcare 11030395. PMID: 36766971; PMCID: PMC9914786.
54. Beynen, Anton. (2017). Beynen A C, 2017. Flax in dog food. 10.13140/RG.2.2.21017.93283.
55. Lindinger, Michael. (2019). Ground Flaxseed—How Safe is it for Companion Animals and for Us? Veterinary Science Research. 1. 10.30564/vsr.v1i1.1158.
56. Beer C, Wood S, Veghte R H. A randomized, double-blind, placebo-controlled clinical trial to investigate the effect of Cynatine® HNS on skin characteristics. Int J Cosmet Sci. 2013 December; 35(6):608-12. doi: 10.1111/ics.12084. Epub 2013 Sep. 2. PMID: 23902431.
57. Beer C, Wood S, Veghte R H. A randomized, double-blind, placebo-controlled clinical trial to investigate the effect of Cynatine® FLX on symptoms of osteoarthritis. J Diet Suppl. 2013 September; 10(3):184-94. doi: 10.3109/19390211.2013.822449. Epub 2013 Aug. 9. PMID: 23927594.
58. https://www.roxlor.com/cynatineflx.php
59. Martello E, Bigliati M, Adami R, Biasibetti E, Bisanzio D, Meineri G, Bruni N. Efficacy of a dietary supplement in dogs with osteoarthritis: A randomized placebo-controlled, double-blind clinical trial. PLoS One. 2022 Feb. 16; 17(2):e0263971. doi: 10.1371/journal.pone.0263971. PMID: 35171954; PMCID: PMC8849458.
60. Giteru, S. G., Ramsey, D. H., Hou, Y., Cong, L., Mohan, A., & Bekhit, A. E.-D. A. (2023). Wool Keratin as a novel alternative protein: A comprehensive review of extraction, purification, nutrition, safety, and food applications. Comprehensive Reviews in Food Science and Food Safety, 22, 643-687. https://doi.org/10.1111/1541-4337.13087
61. Tayyem R F, Heath D D, Al-Delaimy W K, Rock C L. Curcumin content of turmeric and curry powders. Nutr Cancer. 2006; 55(2):126-31. doi: 10.1207/s15327914nc5502_2. PMID: 17044766.
62. Tayyem R F, Heath D D, Al-Delaimy W K, Rock C L. Curcumin content of turmeric and curry powders. Nutr Cancer. 2006; 55(2):126-31. doi: 10.1207/s15327914nc5502_2. PMID: 17044766.
63. Rees, C. A. & Bauer, John & Burkholder, W. J. & Kennis, R. A. & Dunbar, B. L. & Bigley, K. (2001). Effects of dietary flax seed and sunflower seed supplementation on normal canine serum polyunsaturated fatty acids and skin and hair coat condition scores. Veterinary dermatology. 12. 111-7. 10.1046/j.1365-3164.2001.00234.x.
64. Reichling, J & Schmökel, Hugo & Fitzi, Julika & Bucher, Stephan & Saller, Reinhard. (2004). Dietary support with Boswellia resin in canine inflammatory joint and spinal disease. Schweizer Archiv für Tierheilkunde. 146. 71-9. 10.1024/0036-7281.146.2.71.
65. Serra Aguado C I, Ramos-Plá J J, Soler C, Segarra S, Moratalla V, Redondo J I. Effects of Oral Hyaluronic Acid Administration in Dogs Following Tibial Tuberosity Advancement Surgery for Cranial Cruciate Ligament Injury. Animals (Basel). 2021 Apr. 27; 11(5):1264. doi: 10.3390/ani11051264. PMID: 33925642; PMCID: PMC8146498.
66. Borchers A, Epstein S E, Gindiciosi B, Cartoceti A, Puschner B. Acute enteral manganese intoxication with hepatic failure due to ingestion of a joint supplement overdose. Journal of Veterinary Diagnostic Investigation. 2014; 26(5):658-663. doi:10.1177/1040638714544316
67. Murai T, Kawasumi K, Tominaga K, Okada Y, Kobayashi M, Arai T. Effects of Astaxanthin supplementation in healthy and obese dogs. Vet Med (Auckl). 2019 Feb. 15; 10:29-35. doi: 10.2147/VMRR.S186202. PMID: 30859086; PMCID: PMC6385744.
68. Park J S, Kim H W, Mathison B D, Hayek M G, Massimino S, Reinhart G A, Chew B P. Astaxanthin uptake in domestic dogs and cats. Nutr Metab (Lond). 2010 Jun. 21; 7:52. doi: 10.1186/1743-7075-7-52. PMID: 20565958; PMCID: PMC2898833.
69. Beynen, Anton. (2016). Chelates in dog foods. 10.13140/RG.2.2.28134.63045.
70. Barbeau-Grégoire M, Otis C, Cournoyer A, Moreau M, Lussier B, Troncy E. A 2022 Systematic Review and Meta-Analysis of Enriched Therapeutic Diets and Nutraceuticals in Canine and Feline Osteoarthritis. Int J Mol Sci. 2022 Sep. 8; 23(18):10384. doi: 10.3390/ijms231810384. PMID: 36142319; PMCID: PMC9499673.
71. Erena Gil-Quintana, Andrea Molero and Andr&es Aguirre. “OvopetÂ® a new and effective treatment to decrease inflammation, pain and lameness in competing trotters.” Journal of Veterinary Medicine and Animal Health 12, no. 1 (2020): 1-6.
72. Mukhopadhyay N, Shukla A, Makhal P N, Kaki V R. Natural product-driven dual COX-LOX inhibitors: Overview of recent studies on the development of novel anti-inflammatory agents. Heliyon. 2023 Mar. 14; 9(3):e14569. doi: 10.1016/j.heliyon.2023.e14569. PMID: 37020932; PMCID: PMC10068128.
73. Alluri V K, Kundimi S, Sengupta K, Golakoti T, Kilari E K. An Anti-Inflammatory Composition of Boswellia serrata Resin Extracts Alleviates Pain and Protects Cartilage in Monoiodoacetate-Induced Osteoarthritis in Rats. Evid Based Complement Alternat Med. 2020 May 21; 2020:7381625. doi: 10.1155/2020/7381625. PMID: 32565872; PMCID: PMC7261341.
74. https://www.verywellhealth.com/oats-avena-sativa-4707610
75. Lindsay K. Caesar and Nadja B. Cech (2019): Synergy and antagonism in natural product extracts. Natural Product Reports.
76. https://pubs.rsc.org/en/content/articlelanding/2019/np/c9np00011a#cit7
77. Ilya Raskin 1, Christophe Ripoll (2004). Pub Med. Can an apple a day keep the doctor away?
78. Katherine H M Cox, Andrew Pipingas, and Andrew B Scholey (2014), Journal of
79. Psychopharmacology. https://journals.sagepub.com/doi/abs/10.1177/0269881114552744.
80. National Institutes of Health (NIH) (.gov). https://www.ncbi.nlm.nih.gov>articles>PMC5641835 by M Clapp⋅2017⋅Cited by 586—Increasing evidence has associated gut microbiota to both gastrointestinal and extra gastrointestinal diseases. Dysbiosis and inflammation of the . . .
81. NPR https://www.npr.org>health-shots 2023/07/08>stud . . . the gut microbiome and mental health from www.npr.org Jul. 8, 2023—Several studies have shown that fecal transplants rich with bacteria from depressed rats, as well as from depressed humans, can induce . . .
82. Loma Linda University. https://news.llu.edu Health & Wellness the gut microbiome and mental health from news.llu.edu Jul. 19, 2023—“Emerging evidence suggests that imbalances in the gut microbiota can disrupt this communication, potentially leading to mental health disorders . . .
83. Frontiers https://www.frontiersin.org>fpsyg.2023.1215674>full by G D Valle⋅2023⋅Cited by 14—Alterations in the human gut microbiota composition have been linked with mood disorders and neuropsychiatric disorders (Huang et al., 2019) and, in turn, with . . .
84. The Washington Post https://www.washingtonpost.com>wellness>2023/01/31 the gut microbiome and mental health from www.washingtonpost.com Jan. 31, 2023—Research has long suggested a link between our diet and our mental health. The gut microbiome—the collective genome of trillions of bacteria . . .
85. https://pubmed.ncbi.nlm.nih.gov/25776839/
86. https://pubmed.ncbi.nlm.nih.gov/37248896/#:˜:text=Boswellic%20Acids%20Improve%20Clinical%20Cognitive,Mild%20to%20Moderate%20Alzheimer's%20Disease
87. https://www.ergo-log.com/omega-3-fatty-acids-more-effective-when-taken-with-Lecithin.html
88. https://pubmed.ncbi.nlm.nih.gov/25776839/
89. https://www.ncbi.nlm.nih.gov/pmc/articles/PMC10384867/).
90. https://pubmed.ncbi.nlm.nih.gov/37248896/#:˜:text=Boswellic%20Acids%20Improve%20Clinical%20Cognitive,Mild%20to%20Moderate%20Alzheimer's%20Disease

What is claimed is:

1. A synergistic phytomedical formulation for mind and body wellbeing, comprising the following concentrations:
a Stabilized Flax Meal of between 250 mg and 500 mg;
a Cynatine® FLX of between 100 mg and 250 mg;
a Sunflower Lecithin of between 50 mg and 100 mg; a
Curcumin C-3 Reduct® of between 40 mg and 90 mg;
a Boswellia extract of between 20 mg and 70 mg;
an Egg Shell Membrane of between 20 mg and 70 mg;
a Neuravena®-Green Oat Extract of between 30 mg and 200 mg;
a Hyaluronic Acid of between of between 3 mg and 7 mg; a
Manganese Chelate of between 2 mg and 5 mg, and
an Astaxanthin-AstaReal® of between 1 mg and 3 mg.
wherein said formulation is effective in inhibiting Cyclooxygenase (COX) and Lipoxygenase (LOX) pathways, promoting muscle recovery, cartilage remodeling, and reducing discomfort, while additionally addressing mental and cognitive states by reducing anxiety and stress.

2. The synergistic phytomedical formulation of claim 1, wherein the preferred concentration comprises: Stabilized Flax Meal between 400 mg, Cynatine® FLX 150 mg, Sunflower Lecithin 80 mg, Curcumin C-3 Reduct® 50 mg, Boswellia extract 50 mg, Egg Shell Membrane 50 mg, Green Oat Extract-Neuravena® 50 mg, Hyaluronic Acid 5 mg, Manganese Chelate 3 mg, and Astaxanthin-AstaReal® 2 mg.

3. The synergistic phytomedical formulation of claim 2, wherein the Cynatine FLX is derived from solubilized Keratin protein obtained from sheep's wool, exhibits potent anti-inflammatory and antioxidant properties, thereby supporting joint health and reducing symptoms of osteoarthritis.

4. The synergistic phytomedical formulation of claim 1, wherein the Stabilized Flax Meal provides essential nutritive properties, bioactive compounds and microbiome (second brain) support including Omega-3 fatty acids and Lignans, to promote overall health and enhance the anti-inflammatory properties of the formulation.

5. The synergistic phytomedical formulation of claim 4, wherein the Stabilized Flaxseed Meal is formulated to build joint resilience, reduces inflammation, improve mobility and reduce pain, protect the joints, improve gut health (microbiome), promotes heart health, lower risk of obesity, diabetes, malignancy, improved cognition/mood and to promote wound healing.

6. The synergistic phytomedical formulation of claim 2, wherein the Cynatine® FLX acts as a primary defense against oxidative damage, protect and rebuild damaged joints, contains the building blocks needed for joint repair and tissue remodeling, supports healthy epithelia of skin, promotes skeletal muscle functions and reduces inflammation.

7. The synergistic phytomedical formulation of claim 2, wherein the Sunflower Lecithin acts as an emulsifier to increase bioavailability and enhance the efficacy of the formulation, while also providing essential fatty acids and supporting memory, learning, mood, and immune functions as well as improving skin and coat quality

8. The synergistic phytomedical formulation of claim 3, wherein the Boswellia Extract exhibits anti-inflammatory properties, reducing lameness and local pain, improving joint mobility, and providing cartilage protection.

9. The synergistic phytomedical formulation of claim 3, wherein the Egg Shell Membrane contributes collagen, glucosamine, and hyaluronic acid to promote joint health, reduce joint pain, and increase joint function.

10. The synergistic phytomedical formulation of claim 2, wherein the Green Oat Extract-Neuravena® is formulated to promote calming effects, improve memory, cognition, mood, promote gut health, improve skin condition, increase energy levels, regulate fat levels, reduce risk of type 2 diabetes and regulate blood pressure.

11. The synergistic phytomedical formulation of claim 2, wherein the Hyaluronic Acid improves joint mobility, synovial fluid production and promotes rebuilding of joints.

12. The synergistic phytomedical formulation of claim 2, wherein the Manganese Chelate improves the absorption of nutrients into the system.

13. The synergistic phytomedical formulation of claim 2, wherein the Astaxanthin-AstaReal® acts as an anti-oxidant/free radical fighter, neuro protective and is suggested to help inhibit the growth of certain types of tumors, reduces the risk of certain chronic diseases such as obesity, hypertension and type 2 diabetes.

14. A synergistic phytomedical formulation for mind and body wellbeing, comprising the following concentrations Stabilized Flax Meal between 400 mg, Cynatine® FLX150 mg, Sunflower Lecithin 80 mg, Curcumin C-3 Reduct® 50 mg, Boswellia Extract 50 mg, Egg Shell Membrane 50 mg, Neuravena®-Green Oat Extract 50 mg, Hyaluronic Acid 5 mg, Manganese Chelate 3 mg, and Astaxanthin-AstaReal® 2 mg, wherein; the active ingredients dosage is formulated with safety levels of:
Dogs of <11.3 kg (<25 lb.): ¼ chew, 11.3 kg-22.7 kg (25-50 lb.): ½ chew, 22.7 kg-34 kg (50-75 lb.): 1 chew, and >34 kg (>75 lb.): 2 chews.

15. The synergistic phytomedical formulation of claim 14, wherein the Flaxseed Meal safety levels for Small Dogs and Cats is 0.125 g/kg, big dogs: 0.23 g/kg and each chew consist of 400 mg of Stabilized Flaxseed Meal.

16. The synergistic phytomedical formulation of claim 14, wherein:
the Cynatine® FLX is safe in humans and as human food as a high-value protein ingredient that supports normal body functions, wherein one chew contains 150 mg of Cynatine® FLX efficacy levels at the factory recommendation is 150 mg to 200 mg of Cynatine® FLX for a dog weighing 70 lbs., and human effective levels in 6 to 8 days and in dogs they predict Cynatine FLX to show effects in 30 to 60 days;
Curcumin C-3 Reduct® safety levels are 50 mg to 250 mg of Curcumin 3 times a day;
3.14% of pure Turmeric powder is Curcumin, each chew contains 50 mg of stabilized Tetrahydrocurcuminoids as safe levels in the product;
Sunflower Lecithin safety levels are 1000-10,000 mg/kg and each chew contain 80 mg of Sunflower Lecithin with safe levels in the product is safe;
Boswellia Extract safety levels are 400 mg per 10 kg body weight once a day and each chew contain 50 mg of Boswellia Extract as safe levels present in the formula of Egg Shell Membrane safety levels are 13.5 mg/kg once a day and each chew contain 50 mg of Egg Shell Membranes as safe levels in the formula;
Hyaluronic Acid safety levels are up to 26 kg body weight: 27 mg of HA per day;
26 kg body weight: 54 mg of HA per day and each chew contains 5 mg of HA in the formula is safe;
Manganese Chelate safety levels are 0.16 mg/kg/day with an absorption efficacy of 10% and each chew contains 3 mg of Manganese Chelate safe amounts in formula, Astaxanthin-AstaReal® is 0.3 mg/kg/day of efficacy levels with no toxicity levels have been determined, and
Neuravena-Green Oat Extract® of between 30 mg and 200 mg;
Wherein each of these active ingredients has no noted adverse interaction with any of the other active ingredients in the formula

17. A method of formulating a synergistic phytomedical for therapeutic intervention, comprising steps of:
a. administering said formulation to a subject in need thereof;
b. adding a formula for reducing inflammation, discomfort, stress and anxiety;
c. providing a formula for promoting muscle recovery, cartilage remodeling, microbiome (second brain) health and overall joint health, and
d. incorporating a formula for improving overall mental and cognitive functions.

18. The method of claim 17, wherein the formulation further comprises a specific dual approach to pain and inflammation in dogs designed to address the anti-inflammation support and the equally important stressed mental state caused by chronic pain and causes a slower healing process.

19. The method of claim 18, wherein the formulation further comprises step of:
a. adding a composition that is formulated for fighting the mental stress caused by injury (pain and inflammation);
b. incorporating a formula designed for improving mental state and mood (wellbeing) as a precursor and equal partner in faster, more targeted healing and mind and body wellbeing (CNS and ENS), and
c. incorporating a synergistic formula for addressing inflamed joints and tissues and all with a non-pharmaceutical, natural botanically derived delivery vehicle.
